SEQUENCE LISTING (28 sequences)

SEQ ID NO: 1 - protein, 5 aa, Artificial Sequence
  Description of Artificial Sequence Synthetic peptide
SYAMS

SEQ ID NO: 2 - protein, 16 aa, Artificial Sequence
  Description of Artificial Sequence Synthetic peptide
SISKGGNTYYADSVKG

SEQ ID NO: 3 - protein, 10 aa, Artificial Sequence
  Description of Artificial Sequence Synthetic peptide
GWGDYGWFAY

SEQ ID NO: 4 - protein, 7 aa, Artificial Sequence
  Description of Artificial Sequence Synthetic peptide
GFTFSSY

SEQ ID NO: 5 - protein, 5 aa, Artificial Sequence
  Description of Artificial Sequence Synthetic peptide
SKGGN

SEQ ID NO: 6 - protein, 10 aa, Artificial Sequence
  Description of Artificial Sequence Synthetic peptide
GWGDYGWFAY

SEQ ID NO: 7 - protein, 8 aa, Artificial Sequence
  Description of Artificial Sequence Synthetic peptide
GFTFSSYA

SEQ ID NO: 8 - protein, 7 aa, Artificial Sequence
  Description of Artificial Sequence Synthetic peptide
ISKGGNT

SEQ ID NO: 9 - protein, 13 aa, Artificial Sequence
  Description of Artificial Sequence Synthetic peptide
ARGWGDYGWFAYW

SEQ ID NO: 10 - protein, 10 aa, Artificial Sequence
  Description of Artificial Sequence Synthetic peptide
GFTFSSYAMS

SEQ ID NO: 11 - protein, 9 aa, Artificial Sequence
  Description of Artificial Sequence Synthetic peptide
SISKGGNTY

SEQ ID NO: 12 - protein, 10 aa, Artificial Sequence
  Description of Artificial Sequence Synthetic peptide
GWGDYGWFAY

SEQ ID NO: 13 - protein, 11 aa, Artificial Sequence
  Description of Artificial Sequence Synthetic peptide
KASQDINRYLN

SEQ ID NO: 14 - protein, 7 aa, Artificial Sequence
  Description of Artificial Sequence Synthetic peptide
RANRLLD

SEQ ID NO: 15 - protein, 9 aa, Artificial Sequence
  Description of Artificial Sequence Synthetic peptide
LQYDEFPLT

SEQ ID NO: 16 - protein, 11 aa, Artificial Sequence
  Description of Artificial Sequence Synthetic peptide
KASQDINRYLN

SEQ ID NO: 17 - protein, 7 aa, Artificial Sequence
  Description of Artificial Sequence Synthetic peptide
RANRLLD

SEQ ID NO: 18 - protein, 9 aa, Artificial Sequence
  Description of Artificial Sequence Synthetic peptide
LQYDEFPLT

SEQ ID NO: 19 - protein, 6 aa, Artificial Sequence
  Description of Artificial Sequence Synthetic peptide
QDINRY

SEQ ID NO: 20 - protein, 3 aa, Artificial Sequence
  Description of Artificial Sequence Synthetic peptide
RAN

SEQ ID NO: 21 - protein, 9 aa, Artificial Sequence
  Description of Artificial Sequence Synthetic peptide
LQYDEFPLT

SEQ ID NO: 22 - protein, 11 aa, Artificial Sequence
  Description of Artificial Sequence Synthetic peptide
KASQDINRYLN

SEQ ID NO: 23 - protein, 7 aa, Artificial Sequence
  Description of Artificial Sequence Synthetic peptide
RANRLLD

SEQ ID NO: 24 - protein, 9 aa, Artificial Sequence
  Description of Artificial Sequence Synthetic peptide
LQYDEFPLT

SEQ ID NO: 25 - protein, 118 aa, Artificial Sequence
  Description of Artificial Sequence Synthetic polypeptide
QVQLVESGGGVVQPGRSLRLSCAASGFTFSSYAMSWVRQAPGKGLEWVASISKGGNTYYADSVKGRFTISRDNSKNTLYLQMNSLRAEDTAVYYCARGWGDYGWFAYWGQVTLVTVSS

SEQ ID NO: 26 - protein, 107 aa, Artificial Sequence
  Description of Artificial Sequence Synthetic polypeptide
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIK

SEQ ID NO: 27 - protein, 448 aa, Artificial Sequence
  Description of Artificial Sequence Synthetic polypeptide
QVQLVESGGGVVQPGRSLRLSCAASGFTFSSYAMSWVRQAPGKGLEWVASISKGGNTYYADSVKGRFTISRDNSKNTLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, Artificial Sequence
  Description of Artificial Sequence Synthetic polypeptide
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0127345 A1
(43) Pub. Date: Apr. 28, 2022

(54) Methods of Reducing Tau in Human Subjects

(71) Applicant: JANSSEN PHARMACEUTICA NV, Beerse (BE)

(72) Inventors: Wendy R. Galpern, Waltham, MA (US); Maarten Timmers, Bilzen (BE); Tom Lieven K. Jacobs, Kontich (BE); Partha Nandy, Titusville, NJ (US)

(21) Appl. No.: 17/509,889

(22) Filed: Oct. 25, 2021

Related U.S. Application Data
(60) Provisional application No. 63/105,804, filed on Oct. 26, 2020.

(51) Int. Cl. C07K 16/18; A61P 25/28

(52) U.S. Cl. CPC C07K 16/18; A61P 25/28

(57) ABSTRACT

Methods are described for reducing tau in a subject. The methods involve administering to a subject an anti-tau antibody that binds to tau, in particular that bind to a phosphorylated epitope on tau. The methods may be used to reduce p217+tau in CSF, including total p217+tau and free p217+tau.

### CROSS-REFERENCE TO RELATED APPLICATIONS

This application claims the benefit of U.S. Provisional Application No. 63/105,804, filed on Oct. 26, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 6, 2021, is named JNJ_009_US1-SL.txt and is 13,777 bytes in size.

### FIELD OF THE INVENTION

The present invention is in the field of medical treatment. In particular, the invention relates to anti-tau antibodies and their use in reducing tau in human subjects.

### BACKGROUND

Alzheimer's Disease is a neurodegenerative disease characterized by cognitive deficits and memory loss, as well as behavioral and psychiatric symptoms that include anxiety, depression, and agitation. This disease is associated with aging and is believed to represent the fourth most common medical cause of death in the United States.

The hallmark pathological features of Alzheimer's Disease are amyloid plaques and neurofibrillary tangles. Amyloid plaques primarily consist of beta-amyloid (Aβ). Many therapies currently in development aimed at modifying or slowing the progression of Alzheimer's Disease are targeting Aβ. Such therapies include Eli Lilly's solanezumab, Biogen's aducanumab, and Roche's crenezumab, which are all humanized monoclonal antibodies against Aβ.

Neurofibrillary tangles consist of aggregates of hyperphosphorylated tau protein and are generally found in several areas of the human brain of patients with Alzheimer's Disease that are important for memory and cognitive function. The main physiological function of tau is microtubule polymerization and stabilization. The binding of tau to microtubules occurs by ionic interactions between positive charges in the microtubule binding region of tau and negative charges on the microtubule lattice (Butner and Kirschner 1991). Tau protein contains 85 possible phosphorylation sites and phosphorylation at many of these sites interferes with the primary function of tau. Tau that is bound to the axonal microtubule lattice is in a hypo-phosphorylation state, while aggregated tau in Alzheimer's Disease is hyper-phosphorylated.

Several candidate drugs that prevent or clear tau aggregation are currently in development (Brunden et al. 2009). Studies in transgenic mice models have shown that both active and passive tau immunization can have beneficial therapeutic effects (Asuni et al. 2007; Boutajangout et al. 2011). Further, activity has been reported with both phospho-directed and non-phospho-directed antibodies (Schroeder et al. 2016). However, a mechanistic understanding of the efficacy and safety of the various approaches is not well established (Sigurdsson 2016).

There remains a need for effective therapeutics that prevent tau aggregation and tauopathy progression to treat tauopathies such as Alzheimer's Disease.

### SUMMARY OF THE INVENTION

Some of the main aspects of the present invention are summarized below. Additional aspects are described in the Detailed Description of the Invention, Example, and Claims sections of this disclosure. The description in each section of this disclosure is intended to be read in conjunction with the other sections. Furthermore, the various embodiments described in each section of this disclosure can be combined in various ways, and all such combinations are intended to fall within the scope of the present invention.

Accordingly, the disclosure provides methods of reducing tau in subjects, preferably phosphorylated tau.

One aspect of the invention relates to a method of reducing total cerebrospinal fluid p217+tau in a subject in need thereof, the method comprising administering to the subject a composition comprising a pharmaceutically acceptable carrier and about 1 mg/kg to about 60 mg/kg per dose of a monoclonal antibody.

One aspect relates to a method of reducing free cerebrospinal fluid p217+tau in a subject in need thereof, the method comprising administering to the subject a composition comprising a pharmaceutically acceptable carrier and about 1 mg/kg to about 60 mg/kg per dose of a monoclonal antibody.

Another aspect relates to a method of reducing total cerebrospinal fluid tau in a subject in need thereof, the method comprising administering to the subject a composition comprising a pharmaceutically acceptable carrier and about 1 mg/kg to about 60 mg/kg per dose of a monoclonal antibody.

A further aspect relates to a method of reducing cerebrospinal fluid p181tau in a subject in need thereof, the method comprising administering to the subject a composition comprising a pharmaceutically acceptable carrier and about 1 mg/kg to about 60 mg/kg per dose of a monoclonal antibody.

The monoclonal antibody for use in the methods of the present invention may comprise: a heavy chain variable complementarity-determining region (CDR) 1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15. In some embodiments, the monoclonal antibody comprises: a heavy chain variable CDR1 having the amino acid sequence of SEQ ID NO: 1, a heavy chain variable CDR2 having the amino acid sequence of SEQ ID NO: 2, a heavy chain variable CDR3 having the amino acid sequence of SEQ ID NO: 3, a light chain variable CDR1 having the amino acid sequence of SEQ ID NO: 13, a light chain variable CDR2 having the amino acid sequence of SEQ ID NO: 14, and a light chain variable CDR3 having the amino acid sequence of SEQ ID NO: 15.

The monoclonal antibody may comprise a heavy chain variable region comprising the amino acid sequence of SEQ ID NO: 25, and a light chain variable region comprising the amino acid sequence of SEQ ID NO: 26. In certain embodiments, the monoclonal antibody comprises a heavy chain variable region having the amino acid sequence of SEQ ID NO: 25, and a light chain variable region having the amino acid sequence of SEQ ID NO: 26.

Further, the monoclonal antibody may comprise a heavy chain comprising the amino acid sequence of SEQ ID NO: 27, and a light chain comprising the amino acid sequence of SEQ ID NO: 28. In certain embodiments, the monoclonal antibody comprises a heavy chain having the amino acid sequence of SEQ ID NO: 27, and a light chain having the amino acid sequence of SEQ ID NO: 28.

In addition to the monoclonal antibody, the composition may contain histidine, sucrose, polysorbate 20, and ethylenediamine tetra-acetic acid. The composition may have a pH of about 5-6.

In some embodiments, the methods of the invention may comprise administering to the subject the composition comprising about 10 mg/kg to about 40 mg/kg, or about 20 mg/kg to about 60 mg/kg, or about 40 mg/kg to about 60 mg/kg, per dose of the monoclonal antibody. In certain embodiments, the methods may comprise administering to the subject the composition comprising about 1 mg/kg, 3 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, or any value in between, per dose of the monoclonal antibody.

The composition may be administered subcutaneously or by intravenous infusion. Further, the composition may be administered as more than one dose, for example, as more than one dose in which each dose is separated by a period of about 4 weeks.

Administration of the monoclonal antibody may result in a median serum Tₘₐₓ of the monoclonal antibody of about 0.05 days to about 0.25 days after administration. In addition, or alternatively, administration of the monoclonal antibody may result in a median serum t_{1/2} of the monoclonal antibody of about 18 days to about 27 days after administration.

In the methods of the present invention, the subject may be in need of a treatment of Alzheimer's Disease. In particular embodiments, the subject may be in need of a treatment of early Alzheimer's Disease, prodromal Alzheimer's Disease, or mild Alzheimer's Disease.

### BRIEF DESCRIPTION OF THE FIGURES

FIG. 1 shows linear (Panel A) and semi-logarithmic (Panel B) mean serum concentration-time profiles of the anti-tau antibody after administration of single IV dose of the anti-tau antibody in healthy subjects, from Part 1 of the study described in the Example and according to embodiments of the invention.

FIG. 2 shows linear (Panel A) and semi-logarithmic (Panel B) mean CSF concentration-time profiles of the anti-tau antibody after administration of single IV dose of the anti-tau antibody in healthy subjects, from Part 1 of the study described in the Example and according to embodiments of the invention.

FIG. 3 shows individual serum (Panel A) and CSF (Panel B) concentration-time profiles of the anti-tau antibody after administration of single IV dose of the anti-tau antibody in healthy subjects, from Part 1 of the study described in the Example and according to embodiments of the invention.

FIG. 4 shows linear (Panel A) and semi-logarithmic (Panel B) mean serum concentration-time profiles of the anti-tau antibody after administration of the first (Day 1) IV dose of the anti-tau antibody in healthy subjects and subjects with prodromal or mild Alzheimer's Disease, from Part 2 of the study described in the Example and according to embodiments of the invention.

FIG. 5 shows linear (Panel A) and semi-logarithmic (Panel B) mean serum concentration-time profiles of the anti-tau antibody after administration of the third (Day 57) IV dose of the anti-tau antibody in healthy subjects and subjects with prodromal or mild Alzheimer's Disease, from Part 2 of the study described in the Example and according to embodiments of the invention.

FIG. 6 shows linear (Panel A) and semi-logarithmic (Panel B) mean CSF concentration-time profiles of the anti-tau antibody after administration of first (Day 1), second (Day 29), and third (Day 57) IV doses of the anti-tau antibody in healthy subjects and subjects with prodromal or mild Alzheimer's Disease, from Part 2 of the study described in the Example and according to embodiments of the invention. Asterisk (*) indicates that 30 mg/kg cohort not shown in figure as n=2.

FIG. 7 shows individual serum (Panel A) and CSF (Panel B) concentration-time profiles of the anti-tau antibody after administration of first (Day 1), second (Day 29), and third (Day 57) IV doses of the anti-tau antibody in healthy subjects and subjects with prodromal or mild Alzheimer's Disease, from Part 2 of the study described in the Example and according to embodiments of the invention.

FIG. 8 shows change in CSF free p217+tau, as percent of baseline, after administration of single IV dose of the anti-tau antibody in healthy subjects, from Part 1 of the study described in the Example and according to embodiments of the invention. Graph shows group mean+/−standard deviation for each cohort.

FIG. 9 shows change in CSF total p217+tau, as percent of baseline, after administration of single IV dose of the anti-tau antibody in healthy subjects, from Part 1 of the study described in the Example and according to embodiments of the invention. Graph shows group mean+/−standard deviation for each cohort.

FIG. 10 shows CSF free p217+tau, as percent of baseline, after administration of multiple IV doses of the anti-tau antibody in healthy subjects and subjects with prodromal or mild Alzheimer's Disease, from Part 2 of the study described in the Example and according to embodiments of the invention. Graph shows group mean+/−standard deviation for each cohort.

FIG. 11 shows CSF total p217+tau, as percent of baseline, after administration of multiple IV doses of the anti-tau antibody in healthy subjects and subjects with prodromal or mild Alzheimer's Disease, from Part 2 of the study described in the Example and according to embodiments of the invention. Graph shows group mean+/−standard deviation for each cohort.

FIG. 12 shows CSF total tau, as percent of baseline, after administration of multiple IV doses of the anti-tau antibody in healthy subjects and subjects with prodromal or mild Alzheimer's Disease, from Part 2 of the study described in the Example and according to embodiments of the invention. Graph shows group mean+/−standard deviation for each cohort.

FIG. 13 shows CSF total p181tau, as percent of baseline, after administration of multiple IV doses of the anti-tau antibody in healthy subjects and subjects with prodromal or mild Alzheimer's Disease, from Part 2 of the study described in the Example and according to embodiments of the invention. Graph shows group mean+/−standard deviation for each cohort.

FIG. 14 shows maximum CSF total tau change, as percent of baseline, versus CSF p217+tau/total tau concentration ratio in all subjects administered multiple IV doses of the anti-tau antibody, from Part 2 of the study described in the Example and according to embodiments of the invention. Linear regression indicates a non-zero slope with p<0.0001.

FIG. 15 shows maximum CSF p181tau change, as percent of baseline, versus CSF p217+tau/p181tau concentration ratio in all subjects administered multiple IV doses of the anti-tau antibody, from Part 2 of the study described in the Example and according to embodiments of the invention. Linear regression indicates a non-zero slope with p<0.0001.

### DETAILED DESCRIPTION OF THE INVENTION

The practice of the present invention will employ, unless otherwise indicated, conventional techniques of immunology, pharmaceutics, formulation science, cell biology, molecular biology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10. Where a numeric term is preceded by “about,” the term includes the stated number and values ±10% of the stated number.

As used herein, the term “antibody” or “immunoglobulin” is used in a broad sense and includes immunoglobulin or antibody molecules including polyclonal antibodies, monoclonal antibodies including murine, human, human-adapted, humanized, and chimeric monoclonal antibodies and antibody fragments. In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa and lambda, based on the amino acid sequences of their constant domains.

In addition to the heavy and light constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat numbering scheme: “Complementarity Determining Regions” or “CDRs” are based on sequence variability (Wu and Kabat 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL)).
(ii) Chothia numbering scheme: The term “hypervariable region,” “HVR” or “HV” refers to the regions of an antibody variable domain which are hypervariable in structure as defined by Chothia and Lesk (Chothia and Lesk 1987). Generally, the antigen-binding site has three hypervariable regions in each VH (H1, H2, H3) and VL (L1, L2, L3). Numbering systems as well as annotation of CDRs and HVs have been revised by Abhinandan and Martin (Abhinandan and Martin 2008).
(iii) IMGT numbering scheme: Proposed by Lefranc (Lefranc et al. 2003), regions that form the antigen-binding site are defined based on the comparison of V domains from immunoglobulins and T-cell receptors. The International ImMunoGeneTics (IMGT) database provides a standardized numbering and definition of these regions. The correspondence between CDRs, HVs and IMGT delineations is described in Lefranc et al.
(iv) Martin numbering scheme (also known as ABM numbering scheme): A compromise between Kabat and Chothia numbering schemes as described by Martin (Martin 2010).
(v) The antigen-binding site can be delineated based on “Specificity Determining Residue Usage” (SDRU) (Almagro 2004), where SDR, refers to amino acid residues of an immunoglobulin that are directly involved in antigen contact.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g., acetate, phosphate, or citrate buffer), a surfactant (e.g., polysorbate), a stabilizing agent (e.g., polyol or amino acid), a preservative (e.g., sodium benzoate), and/or other conventional solubilizing or dispersing agents.

As used herein, the term “tau” or “tau protein”, also known as microtubule-associated protein tau, MAPT, neurofibrillary tangle protein, paired helical filament (PHF)-tau, MAPTL, or MTBT1, refers to an abundant central and peripheral nervous system protein having multiple isoforms. In the human central nervous system (CNS), six major tau isoforms ranging in size from 352 to 441 amino acids in length exist due to alternative splicing (Hanger et al. 2009). Examples of tau include, but are not limited to, tau isoforms in the CNS, such as the 441-amino acid longest tau isoform (4R2N), also named microtubule-associated protein tau isoform 2, that has four repeats and two inserts, such as the human tau isoform 2 having the amino acid sequence represented in GenBank Accession No. NP_005901.2. Other examples of tau include the 352-amino acid long shortest (fetal) isoform (3RON), also named microtubule-associated protein tau isoform 4, that has three repeats and no inserts, such as the human tau isoform 4 having the amino acid sequence represented in GenBank Accession No. NP_058525.1. Examples of tau also include the “big tau” isoform expressed in peripheral nerves that contains 300 additional residues (exon 4a) (Friedhoff et al. 2000). Examples of tau include a human big tau that is a 758 amino acid-long protein encoded by an mRNA transcript 6762 nucleotides long (NM_016835.4), or isoforms thereof. The amino acid sequence of the exemplified human big tau is represented in GenBank Accession No. NP_058519.3. As used herein, the term “tau” includes homologs of tau from species other than human, such as Macaca Fascicularis (cynomolgus monkey), rhesus monkeys or Pan troglodytes (chimpanzee). As used herein, the term “tau” includes proteins comprising mutations, e.g., point mutations, fragments, insertions, deletions, and splice variants of full-length wild type tau. The term “tau” also encompasses post-translational modifications of the tau amino acid sequence. Post-translational modifications include, but are not limited to, phosphorylation.

As used herein, the term “phosphorylated tau” refers to tau that has been phosphorylated on an amino acid residue at one or more locations of the amino acid sequence of tau. The phosphorylated amino acid residues can be, for example, serine (Ser), threonine (Thr) or tyrosine (Tyr). The site on tau that is phosphorylated is preferably a site that is specifically phosphorylated in neurodegenerative diseases such as Alzheimer's Disease. Examples of sites of phosphorylated tau to which the anti-phosphorylated tau antibody binds include, for example, Tyr18, Thr181, Ser199, Ser202, Thr205, Thr212, Ser214, Thr217, Ser396, Ser404, Ser409, Ser422, Thr427. As used throughout the present application, the amino acid positions are given in reference to the sequence of human microtubule-associated protein tau isoform 2 having the amino acid sequence represented in GenBank Accession No. NP_005901.2. Abnormal phosphorylated tau aggregates readily into insoluble oligomers which are neurotoxic and contribute to neurodegeneration (Goedert et al. 1991). The oligomers progress to tangles of so-called paired helical filaments (PHF) (Alonso et al. 2001). The degree of neurofibrillary tangle pathology has been consistently shown to be correlated to the degree of dementia in AD subjects (Bierer et al. 1995; Braak and Braak 1991; Delacourte 2001).

As used herein, the terms “p181tau”, “p181+tau”, and “p-tau181” are used interchangeably and refer to tau that is phosphorylated at Thr181. Similarly, the terms “p217tau”, “p217+tau”, and “p-tau217” are used interchangeably and refer to tau that is phosphorylated at Thr217. The same nomenclature format can be used to refer to tau that is phosphorylated at different amino acid residues.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

An “effective amount” of a therapy is an amount sufficient to carry out a specifically stated purpose, such as to elicit a desired biological or medicinal response in a subject.

The terms “reduce,” “inhibit,” “block,” and “suppress” are used interchangeably and refer to any statistically significant decrease in occurrence or activity or extent or volume, including full blocking or complete elimination of the occurrence or activity or extent or volume. For example, “inhibition” can refer to a decrease of about 10%, 20%, 30%, 40%, 50%, 60%, 70%, 80%, 90%, or 100% in activity or occurrence. As another example, “reduction” can refer to a decrease of about 10%, 20%, 30%, 40%, 50%, 60%, 70%, 80%, 90%, or 100% in extent or volume.

As used herein, “maximum plasma concentration” or “Cmax” refers to the highest observed concentration of a substance (for example, a monoclonal antibody) in a fluid (serum, plasma, cerebrospinal fluid, etc.) of the body of a mammal after administration of the substance to the mammal.

As used herein, “Tmax” refers to the observed time for reaching the maximum concentration of a substance in a fluid of a mammal after administration of that substance to the mammal (i.e., the observed time for reaching Cmax).

As used herein, “area under the curve” or “AUC” is the area under the curve in a plot of the concentration of a substance in a fluid of the body against time. AUC can be a measure of the integral of the instantaneous concentrations during a time interval and has the units mass*time/volume. AUC is usually provided for a time interval between a starting time t₁ and a finishing time t₂ (e.g., AUCₜ₁₋ₜ₂). If only a single time is indicated (e.g., AUCₜ), it means that the starting time is to and the finishing time is t. As used herein, “AUC₂₄” refers to an AUC over a 24-hour period starting at to; “AUC_{inf}” or “AUC_{∞}” refers to AUC over an infinite time period starting at to; “AUCₗₐₛₜ” refers to AUC over a period starting at to and ending at the time corresponding to the last quantifiable fluid concentration; and AUC_{τ} refers to AUC during a dosing interval (τ=28 days).

As used herein, “half-life” or “t_{1/2}” refers to the time required for half the quantity of a substance administered to a mammal to be metabolized or eliminated from a fluid of the mammal by normal biological processes.

As used herein, “CL” refers to the total systemic clearance of a substance after administration.

As used herein, “Vss” refers to the volume distribution at steady state of a substance after administration.

### Anti-Tau Antibodies

The present invention relates to the use of a monoclonal antibody that binds to tau. The anti-tau antibody can bind to a phosphorylated epitope on tau or bind to a non-phosphorylated epitope on tau.

In some embodiments, the anti-tau antibody can bind to a phosphorylated tau protein at an epitope in the proline rich domain of the tau protein. In certain embodiments, the anti-tau antibody can bind to a phosphorylated tau protein at an epitope comprising phosphorylated Thr181, Thr212 and/or Thr217 residues.

In embodiments of the invention, the anti-tau antibody may comprise heavy chain variable CDRs and light chain variable CDRs as shown in Table 1 below.

TABLE 1
Sequences for the heavy chain variable CDRs and light chain
variable CDRs of the anti-tau antibody.

| | | | |
|---|---|---|---|
| Variable Region | CDR1 | CDR2 | CDR3 |

Kabat numbering scheme

| | | | |
|---|---|---|---|
| Heavy Chain | SYAMS | SISKGGNTYYADSVKG | GWGDYGWFAY |
| | (SEQ ID NO: 1) | (SEQ ID NO: 2) | (SEQ ID NO: 3) |
| Light Chain | KASQDINRYLN | RANRLLD | LQYDEFPLT |
| | (SEQ ID NO: 13) | (SEQ ID NO: 14) | (SEQ ID NO: 15) |

Chothia numbering scheme

| | | | |
|---|---|---|---|
| Heavy Chain | GFTFSSY | SKGGN | GWGDYGWFAY |
| | (SEQ ID NO: 4) | (SEQ ID NO: 5) | (SEQ ID NO: 6) |
| Light Chain | KASQDINRYLN | RANRLLD | LQYDEFPLT |
| | (SEQ ID NO: 16) | (SEQ ID NO: 17) | (SEQ ID NO: 18) |

IMGT numbering scheme

| | | | |
|---|---|---|---|
| Heavy Chain | GFTFSSYA | ISKGGNT | ARGWGDYGWFAYW |
| | (SEQ ID NO: 7) | (SEQ ID NO: 8) | (SEQ ID NO: 9) |
| Light Chain | QDINRY | RAN | LQYDEFPLT |
| | (SEQ ID NO: 19) | (SEQ ID NO: 20) | (SEQ ID NO: 21) |

ABM numbering scheme

| | | | |
|---|---|---|---|
| Heavy Chain | GFTFSSYAMS | SISKGGNTY | GWGDYGWFAY |
| | (SEQ ID NO: 10) | (SEQ ID NO: 11) | (SEQ ID NO: 12) |
| Light Chain | KASQDINRYLN | RANRLLD | LQYDEFPLT |
| | (SEQ ID NO: 22) | (SEQ ID NO: 23) | (SEQ ID NO: 24) |

Thus, according to embodiments of the invention, the anti-tau antibody comprises:
(a) a heavy chain variable CDR1 comprising the amino acid sequence of SEQ ID NOS: 1, 4, 7, or 10;
(b) a heavy chain variable CDR2 comprising the amino acid sequence of SEQ ID NOS: 2, 5, 8, or 11;
(c) a heavy chain variable CDR3 comprising the amino acid sequence of SEQ ID NOS: 3, 6, 9, or 12;
(d) a light chain variable CDR1 comprising the amino acid sequence of SEQ ID NOS: 13, 16, 19, or 22;
(e) a light chain variable CDR2 comprising the amino acid sequence of SEQ ID NOS: 14, 17, 20, or 23; and
(f) a light chain variable CDR3 comprising the amino acid sequence of SEQ ID NOS: 15, 18, 21, or 24.

In some embodiments, the anti-tau antibody comprises:
(a) a heavy chain variable CDR1 having the amino acid sequence of SEQ ID NOS: 1, 4, 7, or 10;
(b) a heavy chain variable CDR2 having the amino acid sequence of SEQ ID NOS: 2, 5, 8, or 11;
(c) a heavy chain variable CDR3 having the amino acid sequence of SEQ ID NOS: 3, 6, 9, or 12;
(d) a light chain variable CDR1 having the amino acid sequence of SEQ ID NOS: 13, 16, 19, or 22
(e) a light chain variable CDR2 having the amino acid sequence of SEQ ID NOS: 14, 17, 20, or 23; and
(f) a light chain variable CDR3 having the amino acid sequence of SEQ ID NOS: 15, 18, 21, or 24.

In certain embodiments, the anti-tau antibody comprises a heavy chain variable CDR1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15. In particular embodiments, the anti-tau antibody comprises a heavy chain variable CDR1 having the amino acid sequence of SEQ ID NO: 1, a heavy chain variable CDR2 having the amino acid sequence of SEQ ID NO: 2, a heavy chain variable CDR3 having the amino acid sequence of SEQ ID NO: 3, a light chain variable CDR1 having the amino acid sequence of SEQ ID NO: 13, a light chain variable CDR2 having the amino acid sequence of SEQ ID NO: 14, and a light chain variable CDR3 having the amino acid sequence of SEQ ID NO: 15.

In embodiments of the invention, the anti-tau antibody comprises a heavy chain variable region comprising the amino acid sequence of SEQ ID NO: 25, and a light chain variable region comprising the amino acid sequence of SEQ ID NO: 26. In certain embodiments, the anti-tau antibody comprises a heavy chain variable region having the amino acid sequence of SEQ ID NO: 25, and a light chain variable region comprising the amino acid sequence of SEQ ID NO: 26.

In embodiments of the invention, the anti-tau antibody is an immunoglobulin G (IgG) antibody. In certain embodiments, the anti-tau antibody is an IgG1 antibody. Alternatively, the anti-tau antibody is an IgG2, IgG3, or IgG4 antibody. In other embodiments, the anti-tau antibody is an IgA, IgD, IgE, or IgM antibody.

In embodiments of the invention, the anti-tau antibody comprises a kappa light chain constant region. In other embodiments, the anti-tau antibody comprises a delta light chain constant region.

In preferred embodiments, the anti-tau antibody is an IgG1 antibody having a kappa light chain constant region.

In embodiments of the invention, the anti-tau antibody comprises a heavy chain comprising the amino acid sequence of SEQ ID NO: 27, and a light chain comprising the amino acid sequence of SEQ ID NO: 28. In certain embodiments, the anti-tau antibody comprises a heavy chain having the amino acid sequence of SEQ ID NO: 27, and a light chain having the amino acid sequence of SEQ ID NO: 28.

In preferred embodiments, the anti-tau antibody is a humanized monoclonal antibody.

Anti-tau antibody of the present invention can be produced by a variety of techniques, for example by the hybridoma method (Köhler and Milstein 1975). Chimeric monoclonal antibodies containing a light chain and heavy chain variable region derived from a donor antibody (typically murine) in association with light and heavy chain constant regions derived from an acceptor antibody (typically another mammalian species such as human) can be prepared by a method disclosed in U.S. Pat. No. 4,816,567. CDR-grafted monoclonal antibodies having CDRs derived from a non-human donor immunoglobulin (typically murine) and the remaining immunoglobulin-derived parts of the molecule being derived from one or more human immunoglobulins can be prepared by techniques known to those skilled in the art such as that disclosed in U.S. Pat. No. 5,225,539. Fully human monoclonal antibodies lacking any non-human sequences can be prepared from human immunoglobulin transgenic mice by techniques referenced in (Lonberg et al. 1994; Fishwild et al. 1996; Mendez et al. 1997). Human monoclonal antibodies can also be prepared and optimized from phage display libraries (Knappik et al. 2000; Krebs et al. 2001; Shi et al. 2010).

In embodiments of the invention, the anti-tau antibody may be formulated in a composition comprising a pharmaceutically acceptable carrier. The composition may also comprise one or more pharmaceutically acceptable excipients, which are well known in the art (see Remington's Pharmaceutical Science 1980). The preferred formulation of the pharmaceutical composition depends on the intended mode of administration and therapeutic application. The pharmaceutically-acceptable carriers can be vehicles commonly used to formulate pharmaceutical compositions for animal or human administration. In addition, the pharmaceutical composition may also include other diluents, adjuvants, or nontoxic, nontherapeutic, non-immunogenic stabilizers, and the like. It will be understood that the characteristics of the carrier, excipient or diluent will depend on the route of administration for a particular application.

In certain embodiments, the composition may comprise one or more stabilizing agents (for example, dextran 40, sucrose, glycine, lactose, mannitol, trehalose, maltose), one or more buffers (for example, acetate, citrate, histidine, lactate, phosphate, Tris), one or more surfactants (for example, polysorbate, sodium lauryl sulfate, polyethylene glycol-fatty acid esters, lecithins), one or more chelators (for example, ethylenediamine tetra-acetic acid (EDTA), edetate sodium), and a carrier (for example, water for injection water, physiological phosphate-buffered saline, Ringer's solutions, dextrose solution, Hank's solution). In preferred embodiments, the composition comprises water for injection, histidine, sucrose, polysorbate 20, and EDTA. The composition may have a pH of about 4 to about 7, or about 5 to about 6, preferably about 5.5.

### Methods of Use

A general aspect of the present invention relates to methods of reducing tau in a subject comprising administering to the subject a composition comprising an anti-tau antibody according to embodiments of the invention.

In some embodiments of the invention, the methods are directed to reducing phosphorylated tau in a subject. In preferred embodiments, the methods are directed to reducing p181tau, and/or are directed to reducing p217+tau.

In some embodiments, the methods are directed to reducing total tau, including total phosphorylated tau (for example, total p181tau and/or total p217+tau). In some embodiments, the methods are directed to reducing free tau, including free phosphorylated tau (for example, free p181tau and/or free p217+tau). As used herein “free” in the context of tau refers to tau that is not bound to an antibody, such as the anti-tau antibody of the present invention.

In some embodiments, the methods are directed to reducing tau, including phosphorylated tau (for example, p181tau and/or p217+tau) in the CSF.

In some embodiments, the methods are directed to reducing fragments of phosphorylated tau, including fragments of p181tau and/or p217+tau, in a subject. In certain embodiments, the methods are directed to reducing fragments of phosphorylated tau, including fragments of p181tau and/or p217+tau, in CSF, serum, or both, of a subject.

The ability to reduce tau in CSF can be determined by testing a sample of the CSF from the subject for the presence of tau (including phosphorylated tau such as p181tau and/or p217+tau). Such testing can be performed using traditional total tau and phosphorylated tau enzyme-linked immunosorbent assays (ELISAs) (for example, Innotest hTauAG), as well as high sensitivity ELISAs developed particularly for measuring p217+tau (see, e.g., U.S. Pat. No. 10,591,492, which is incorporated herein by reference).

According to embodiments of the invention, the composition may be administered in an amount of about 1 mg/kg to about 60 mg/kg per dose of the anti-tau antibody. In some embodiments, the composition may be administered in an amount of about 10 mg/kg to about 40 mg/kg per dose, or about 20 mg/kg to about 60 mg/kg per dose, or about 40 mg/kg to about 60 mg/kg per dose, of the anti-tau antibody. In certain embodiments, the composition may be administered in an amount of about 1 mg/kg, 3 mg/kg, 5 mg/kg, 10 mg/kg, 12.5 mg/kg, 15 mg/kg, 20 mg/kg, 25 mg/kg, 30 mg/kg, 35 mg/kg, 37.5 mg/kg, 40 mg/kg, 45 mg/kg, 50 mg/kg, 55 mg/kg, 60 mg/kg, or any value in between, per dose of the anti-tau antibody.

In embodiments of the invention, the composition may be administered in an amount of about 50 mg to about 5000 mg per dose of the anti-tau antibody. In some embodiments, the composition may be administered in an amount of about 1000 mg to about 3000 mg per dose, or about 2000 mg 5000 mg per dose, or about 3000 mg to about 5000 mg per dose, of the anti-tau antibody. In certain embodiments, the composition may be administered in an amount of about 50 mg, 100 mg, 250 mg, 500 mg, 750 mg, 1000 mg, 1200 mg, 1250 mg, 1400 mg, 1500 mg, 1600 mg, 1750 mg, 1800 mg, 2000 mg, 2200 mg, 2250 mg, 2400 mg, 2500 mg, 2600 mg, 2750 mg, 2800 mg, 3000 mg, 3200 mg, 3250 mg, 3400 mg, 3500 mg, 3600 mg, 3750 mg, 3800 mg, 4000 mg, 4200 mg, 4250 mg, 4400 mg, 4500 mg, 4600 mg, 4750 mg, 4800 mg, or 5000 mg, or any value in between, per dose of the anti-tau antibody.

According to some embodiments, the composition may be administered as more than one dose. In certain embodiments, administration of each dose may be separated by a period of time, for example, about 4 weeks.

The composition comprising the anti-tau antibody can be administered by parenteral, topical, oral, intra-arterial, intracranial, intraperitoneal, intradermal, intranasal, or intramuscular means for prophylactic and/or therapeutic treatment. In certain embodiments, the composition can be administered subcutaneously. In certain embodiments, the composition can be administered by intravenous infusion.

According to some embodiments, the subject is a human subject. In certain embodiments, the subject is a human subject in need of treatment of a neurodegenerative disease, disorder, or condition.

As used herein a “neurodegenerative disease, disorder, or condition” includes any neurodegenerative disease, disorder, or condition known to those skilled in the art in view of the present disclosure. Examples of neurodegenerative diseases, disorders, or conditions include neurodegenerative diseases or disorders caused by or associated with the formation of neurofibrillary lesions, such as tau-associated diseases, disorders or conditions, referred to as tauopathies. According to particular embodiments, the neurodegenerative disease, disorder, or condition includes any of the diseases or disorders that show co-existence of tau and/or amyloid pathologies including, but not is limited to, Alzheimer's Disease, Parkinson's Disease, Creutzfeldt-Jacob disease, Dementia pugilistica, Down('s) Syndrome, Gerstmann-Straussler-Scheinker disease, inclusion body myositis, prion protein cerebral amyloid angiopathy, traumatic brain injury, amyotrophic lateral sclerosis, parkinsonism-dementia complex of Guam, Non-Guamanian motor neuron disease with neurofibrillary tangles, argyrophilic grain dementia, corticobasal degeneration, Dementia in Amyotrophic Lateral Sclerosis, diffuse neurofibrillary tangles with calcification, frontotemporal dementia, preferably frontotemporal dementia with parkinsonism linked to chromosome 17 (FTDP-17), frontotemporal lobar dementia, Hallevorden-Spatz disease, multiple system atrophy, Niemann-Pick disease type C, Pick's disease, progressive subcortical gliosis, progressive supranuclear palsy, Subacute sclerosing panencephalitis, Tangle only dementia, Postencephalitic Parkinsonism, Myotonic dystrophy, chronic traumatic encephalopathy (CTE), Primary age-related Tauopathy (PART), cerebral angiopathy or Lewy body dementia (LBD). According to particular embodiments, the neurodegenerative disease, disorder, or condition is Alzheimer's disease or another tauopathy. According to preferred embodiments, the neurodegenerative disease, disorder, or condition is Alzheimer's Disease.

The clinical course of Alzheimer's Disease can be divided into stages, with progressive patterns of cognitive and functional impairments. The stages can be defined using grading scales known in the art including, for instance, NIA-AA Research Framework (see, e.g., Dubois et al. 2016; Dubois et al. 2014; Jack et al. 2018) and the Clinical Demential Rating scale (see, e.g., Berg 1988), the contents of each of which are hereby incorporated by reference in their entirety.

For example, National Institute on Aging-Alzheimer' s Association (NIA-AA) research framework defines Alzheimer's Disease biologically, by neuropathologic change or biomarkers, and treats cognitive impairment as a symptom/sign of the disease rather than the definition of the disease (see, e.g., Jack et al. 2018, the content of which is incorporated herein by reference). According to the NIA-AA definition, an individual with biomarker evidence of AP deposition alone (abnormal amyloid PET scan or low CSF Aβ42 or Aβ42/Aβ40 ratio) with a normal pathologic tau biomarker would be assigned the label “Alzheimer's pathologic change,” and the term “Alzheimer's Disease” would be applied if both biomarker evidence of AP and pathologic tau are present. The NIA-AA also developed a system for staging severity of Alzheimer's Disease. In particular, under the NIA-AA definition (reproduced from Text Box 2 of Jack et al. 2018, supra):

Definition:
A: Aβ biomarkers determine whether or not an individual is in the Alzheimer's continuum.
T: Pathologic tau biomarkers determine if someone who is in the Alzheimer's continuum has Alzheimer's disease

Staging severity:
(N): Neurodegenerative/neuronal injury biomarkers
(C): Cognitive symptoms
A and T indicate specific neuropathologic changes that define Alzheimer's disease, whereas (N) and (C) are not specific to Alzheimer's disease and are therefore placed in parentheses.

According to preferred embodiments, the neurodegenerative disease, disorder, or condition is early Alzheimer's Disease, prodromal Alzheimer's Disease (Alzheimer's Disease with mild cognitive impairment (MCI)), or mild Alzheimer's Disease (also referred to as mild Alzheimer's Disease dementia).

In some embodiments, the neurodegenerative disease, disorder, or condition is mild to moderate Alzheimer's Disease.

In some embodiments, the subject in need of a treatment is amyloid positive in the brain but does not yet show significant cognitive impairment. The amyloid deposition in the brain can be detected using methods known in the art, such as PET scan, immunoprecipitation mass spectrometry, or other methods (for example, use of CSF biomarkers) (Jacket al. 2018).

In other embodiments, the human subject in need of a treatment has abnormal level of CSF AB amyloid 42 (Aβ42) consistent with Alzheimer's Disease pathology. For example, the subject can have low level of CSF Aβ42 or low Aβ42/Aβ40 ratio consistent with Alzheimer's Disease pathology (see, e.g., Jack et al. 2018, supra).

In embodiments of the invention, IV administration of the composition comprising the anti-tau antibody achieves a median serum Tₘₐₓ of the anti-tau antibody of about 0.05 days to about 0.25 days after administration.

In embodiments of the invention, IV administration of the composition comprising the anti-tau antibody achieves a median serum t_{1/2} of the anti-tau antibody of about 18 days to about 27 days after administration.

In some embodiments, IV administration of the composition comprising about 1 mg/kg of the anti-tau antibody achieves one or more of:
(a) serum Cₘₐₓ of about 21 to about 28 μg/mL;
(b) serum Tₘₐₓ of about 0.05 to about 0.5 days;
(c) serum AUCₗₐₛₜ of about 283 to about 361 μg·day/mL;
(d) serum AUC_{∞} of about 300 to about 378 μg.day/mL;
(e) serum t_{1/2} of about 15 to about 23 days; or
(f) serum CL of about 2.6 to about 3.3 mL/day/kg.

In some embodiments, IV administration of the composition comprising about 1 mg/kg of the anti-tau antibody achieves one or more of:
(a) mean serum Cₘₐₓ of about 24.6 μg/mL;
(b) median serum Tₘₐₓ of about 0.11 days;
(c) mean serum AUCₗₐₛₜ of about 322 μg·day/mL;
(d) mean serum AUC_{∞} of about 339 μg·day/mL;
(e) mean serum t_{1/2} of about 18.9 days; or
(f) mean serum CL of about 2.97 mL/day/kg.

In some embodiments, IV administration of the composition comprising about 3 mg/kg of the anti-tau antibody achieves one or more of:
(a) serum Cₘₐₓ of about 53 to about 72 μg/mL;
(b) serum Tₘₐₓ of about 0.04 to about 0.17 days;
(c) serum AUCₗₐₛₜ of about 685 to about 953 μg·day/mL;
(d) serum AUC_{∞} of about 705 to about 993 μg·day/mL;
(e) serum t_{1/2} of about 15 to about 21 days; or
(f) serum CL of about 3.0 to about 4.2 mL/day/kg.

In some embodiments, IV administration of the composition comprising about 3 mg/kg of the anti-tau antibody achieves one or more of:
(a) mean serum Cₘₐₓ of about 62.7 μg/mL;
(b) median serum Tₘₐₓ of about 0.05 days;
(c) mean serum AUCₗₐₛₜ of about 819 μg·day/mL;
(d) mean serum AUC_{∞} of about 849 μg·day/mL;
(e) mean serum t_{1/2} of about 18.1 days; or
(f) mean serum CL of about 3.6 mL/day/kg.

In some embodiments, IV administration of the composition comprising about 10 mg/kg of the anti-tau antibody achieves one or more of:
(a) serum Cₘₐₓ of about 230 to about 302 μg/mL;
(b) serum Tₘₐₓ of about 0.05 to about 0.33 days;
(c) serum AUCₗₐₛₜ of about 2935 to about 4435 μg·day/mL;
(d) serum AUC₂₈ of about 2947 to about 5177 μg·day/mL;
(e) serum t_{1/2} of about 18 to about 34 days; or
(f) serum CL of about 2.1 to about 3.1 mL/day/kg.

In some embodiments, IV administration of the composition comprising about 10 mg/kg of the anti-tau antibody achieves one or more of:
(a) mean serum Cₘₐₓ of about 266 μg/mL;
(b) median serum Tₘₐₓ of about 0.25 days;
(c) mean serum AUCₗₐₛₜ of about 3685 μg·day/mL;
(d) mean serum AUC₂₈ of about 4062 μg·day/mL;
(e) mean serum t_{1/2} of about 26.4 days; or
(f) mean serum CL of about 2.6 mL/day/kg.

In some embodiments, IV administration of the composition comprising about 30 mg/kg of the anti-tau antibody achieves one or more of:
(a) serum Cₘₐₓ of about 523 to about 625 μg/mL;
(b) serum Tₘₐₓ of about 0.06 to about 0.50 days;
(c) serum AUCₗₐₛₜ of about 5921 to about 9077 μg·day/mL;
(d) serum AUC₂₈ of about 6552 to about 10,076 μg·day/mL;
(e) serum t_{1/2} of about 18 to about 28 days; or
(f) serum CL of about 2.9 to about 4.6 mL/day/kg.

In some embodiments, IV administration of the composition comprising about 30 mg/kg of the anti-tau antibody achieves one or more of:
(a) mean serum Cₘₐₓ of about 574 μg/mL;
(b) median serum Tₘₐₓ of about 0.17 days;
(c) mean serum AUCₗₐₛₜ of about 7499 μg.day/mL;
(d) mean serum AUC₂₈ of about 8314 μg·day/mL;
(e) mean serum t_{1/2} of about 23.1 days; or
(f) mean serum CL of about 3.8 mL/day/kg.

In some embodiments, IV administration of the composition comprising about 60 mg/kg of the anti-tau antibody achieves one or more of:
(a) serum Cₘₐₓ of about 1191 to about 1583 μg/mL;
(b) serum Tₘₐₓ of about 0.06 to about 0.92 days;
(c) serum AUCₗₐₛₜ of about 13,450 to about 22,160 μg·day/mL;
(d) serum AUC₂₈ of about 16,751 to about 24,143 μg·day/mL;
(e) serum t_{1/2} of about 15 to about 29 days; or
(f) serum CL of about 2.4 to about 3.7 mL/day/kg.

In some embodiments, IV administration of the composition comprising about 60 mg/kg of the anti-tau antibody achieves one or more of:
(a) mean serum Cₘₐₓ of about 1387 μg/mL;
(b) median serum Tₘₐₓ of about 0.17 days;
(c) mean serum AUCₗₐₛₜ of about 17,805 μg·day/mL;
(d) mean serum AUC₂₈ of about 20,447 μg·day/mL;
(e) mean serum t_{1/2} of about 21.8 days; or
(f) mean serum CL of about 3.0 mL/day/kg.

In some embodiments, IV administration of the composition comprising about 1 mg/kg of the anti-tau antibody achieves one or more of:
(a) CSF Cₘₐₓ of about 9 to about 18 ng/mL;
(b) CSF Tₘₐₓ of about 14 to about 15 days;
(c) CSF AUC_{Day29} of about 210 to about 386 ng·day/mL; or
(d) CSF AUC_{Day57} of about 259 to about 615 ng·day/mL.

In some embodiments, IV administration of the composition comprising about 1 mg/kg of the anti-tau antibody achieves one or more of:
(a) mean CSF Cₘₐₓ of about 13.4 ng/mL;
(b) median CSF Tₘₐₓ of about 14.9 days;
(c) mean CSF AUC_{Day29} of about 298 ng·day/mL; or
(d) mean CSF AUC_{Day57} of about 437 ng·day/mL.

In some embodiments, IV administration of the composition comprising about 1 mg/kg of the anti-tau antibody achieves one or more of:
(a) CSF/serum concentration ratio about one day after administration of about 0.02% to about 0.06%;
(b) CSF/serum concentration ratio about 14 days after administration of about 0.11% to about 0.32%;
(c) CSF/serum concentration ratio about 28 days after administration of about 0.12% to about 0.29%; or
(d) CSF/serum concentration ratio about 56 days after administration of about 0.06% to about 0.74%.

In some embodiments, IV administration of the composition comprising about 1 mg/kg of the anti-tau antibody achieves one or more of:
(a) mean CSF/serum concentration ratio about one day after administration of about 0.03%;
(b) mean CSF/serum concentration ratio about 14 days after administration of about 0.19%;
(c) mean CSF/serum concentration ratio about 28 days after administration of about 0.19%; or
(d) mean CSF/serum concentration ratio about 56 days after administration of about 0.22%.

In some embodiments, IV administration of the composition comprising about 3 mg/kg of the anti-tau antibody achieves one or more of:
(a) CSF Cₘₐₓ of about 31 to about 74 ng/mL;
(b) CSF Tₘₐₓ of about 13.9 to about 14.1 days;
(c) CSF AUC_{Day29} of about 662 to about 1546 ng·day/mL;
(d) CSF AUC_{Day43} of about 914 to about 2134 ng·day/mL; or
(e) CSF AUCₗₐₛₜ of about 914 to about 2134 ng·day/mL.

In some embodiments, IV administration of the composition comprising about 3 mg/kg of the anti-tau antibody achieves one or more of:
(a) mean CSF Cₘₐₓ of about 52.7 ng/mL;
(b) median CSF Tₘₐₓ of about 14.1 days;
(c) mean CSF AUC_{Day29} of about 1104 ng·day/mL;
(d) mean CSF AUC_{Day43} of about 1524 ng·day/mL; or
(e) mean CSF AUCₗₐₛₜ of about 1524 ng·day/mL.

In some embodiments, IV administration of the composition comprising about 3 mg/kg of the anti-tau antibody achieves one or more of:
(a) CSF/serum concentration ratio about one day after administration of about 0.02% to about 0.08%;
(b) CSF/serum concentration ratio about 14 days after administration of about 0.18% to about 0.43%;
(c) CSF/serum concentration ratio about 28 days after administration of about 0.22% to about 0.40%; or
(d) CSF/serum concentration ratio about 42 days after administration of about 0.16% to about 0.80%.

In some embodiments, IV administration of the composition comprising about 3 mg/kg of the anti-tau antibody achieves one or more of:
(a) mean CSF/serum concentration ratio about one day after administration of about 0.04%;
(b) mean CSF/serum concentration ratio about 14 days after administration of about 0.28%;
(c) mean CSF/serum concentration ratio about 28 days after administration of about 0.30%; or
(d) mean CSF/serum concentration ratio about 42 days after administration of about 0.37%.

In some embodiments, IV administration of the composition comprising about 10 mg/kg of the anti-tau antibody achieves one or more of:
(a) CSF Cₘₐₓ of about 170 to about 182 ng/mL;
(b) CSF Tₘₐₓ of about 13.9 to about 15.0 days; or
(c) CSF AUC_{Day29} of about 2365 to about 6187 ng·day/mL.

In some embodiments, IV administration of the composition comprising about 10 mg/kg of the anti-tau antibody achieves one or more of:
(a) mean CSF Cₘₐₓ of about 176 ng/mL;
(b) median CSF Tₘₐₓ of about 14 days; or
(c) mean CSF AUC_{Day29} of about 4276 ng·day/mL.

In some embodiments, IV administration of the composition comprising about 10 mg/kg of the anti-tau antibody achieves one or more of:
(a) CSF/serum concentration ratio about one day after administration of about 0.02% to about 0.07%;
(b) CSF/serum concentration ratio about 14 days after administration of about 0.16% to about 0.34%; or
(c) CSF/serum concentration ratio about 28 days after administration of about 0.19% to about 0.40%.

In some embodiments, IV administration of the composition comprising about 10 mg/kg of the anti-tau antibody achieves one or more of:
(a) mean CSF/serum concentration ratio about one day after administration of about 0.04%;
(b) mean CSF/serum concentration ratio about 14 days after administration of about 0.24%; or
(c) mean CSF/serum concentration ratio about 28 days after administration of about 0.28%.

In some embodiments, IV administration of the composition comprising about 30 mg/kg of the anti-tau antibody achieves one or more of:
(a) CSF Cₘₐₓ of about 224 to about 570 ng/mL;
(b) CSF Tₘₐₓ of about 13.1 to about 29.0 days;
(c) CSF AUC_{Day43} of about 12,601 to about 18,239 ng·day/mL;
(d) CSF AUC_{Day57} of about 7880 to about 11,436 ng·day/mL;
(e) CSF AUCₗₐₛₜ of about 17,027 to about 21,909 ng·day/mL or
(f) CSF AUCₗₐₛₜ of about 9372 to about 12,146 ng·day/mL.

In some embodiments, IV administration of the composition comprising about 30 mg/kg of the anti-tau antibody achieves one or more of:
(a) mean CSF Cₘₐₓ of about 397 ng/mL;
(b) median CSF Tₘₐₓ of about 14.1 days;
(c) mean CSF AUC_{Day43} of about 15,420 ng·day/mL;
(d) mean CSF AUC_{Day57} of about 9658 ng·day/mL;
(e) mean CSF AUCₗₐₛₜ of about 19,468 ng·day/mL; or
(f) mean CSF AUCₗₐₛₜ of about 10,759 ng·day/mL.

In some embodiments, IV administration of the composition comprising about 30 mg/kg of the anti-tau antibody achieves one or more of:
(a) CSF/serum concentration ratio about one day after administration of about 0.02% to about 0.07%;
(b) CSF/serum concentration ratio about 14 days after administration of about 0.17% to about 0.51%;
(c) CSF/serum concentration ratio about 42 days after administration of about 0.15% to about 1.12%; or
(d) CSF/serum concentration ratio about 70 days after administration of about 0.15% to about 1.28%.

In some embodiments, IV administration of the composition comprising about 30 mg/kg of the anti-tau antibody achieves one or more of:
(a) mean CSF/serum concentration ratio about one day after administration of about 0.04%;
(b) mean CSF/serum concentration ratio about 14 days after administration of about 0.30%;
(c) mean CSF/serum concentration ratio about 42 days after administration of about 0.42%; or
(d) mean CSF/serum concentration ratio about 70 days after administration of about 0.45%.

In some embodiments, IV administration of the composition comprising about 60 mg/kg of the anti-tau antibody achieves one or more of:
(a) CSF Cₘₐₓ of about 637 to about 1585 ng/mL; or
(b) CSF Tₘₐₓ of about 6.9 to about 14.1 days.

In some embodiments, IV administration of the composition comprising about 60 mg/kg of the anti-tau antibody achieves one or more of:
(a) mean CSF Cₘₐₓ of about 1111 ng/mL; or
(b) median CSF Tₘₐₓ of about 11 days.

In some embodiments, administration of the composition comprising about 60 mg/kg of the anti-tau antibody achieves one or more of:
(a) CSF/serum concentration ratio about one day after administration of about 0.02% to about 0.10%;
(b) CSF/serum concentration ratio about 7 days after administration of about 0.09% to about 0.43%; or
(c) CSF/serum concentration ratio about 14 days after administration of about 0.17% to about 0.57%.

In some embodiments, IV administration of the composition comprising about 60 mg/kg of the anti-tau antibody achieves one or more of:
(a) mean CSF/serum concentration ratio about one day after administration of about 0.05%;
(b) mean CSF/serum concentration ratio about 7 days after administration of about 0.20%; or
(c) mean CSF/serum concentration ratio about 14 days after administration of about 0.31%.

In some embodiments, three IV administrations of the composition comprising about 5 mg/kg of the anti-tau antibody, in which each administration is separated by about 28 days, achieves after the first administration one or more of:
(a) serum Cₘₐₓ of about 107 to about 142 μg/mL;
(b) serum Tₘₐₓ of about 0.04 to about 0.33 days; or
(c) serum AUC_{τ} of about 952 to about 1308 μg·day/mL.

In some embodiments, three IV administrations of the composition comprising about 5 mg/kg of the anti-tau antibody, in which each administration is separated by about 28 days, achieves after the first administration one or more of:
(a) mean serum Cₘₐₓ of about 125 μg/mL;
(b) mean serum Tₘₐₓ of about 0.05 days; or
(c) mean serum AUC_{τ} of about 1130 μg·day/mL.

In some embodiments, three IV administrations of the composition comprising about 15 mg/kg of the anti-tau antibody, in which each administration is separated by about 28 days, achieves after the first administration one or more of:
(a) serum Cₘₐₓ of about 292 to about 426 μg/mL;
(b) serum Tₘₐₓ of about 0.05 to about 0.17 days; or
(c) serum AUC_{τ} of about 2656 to about 4316 μg·day/mL.

In some embodiments, three IV administrations of the composition comprising about 15 mg/kg of the anti-tau antibody, in which each administration is separated by about 28 days, achieves after the first administration one or more of:
(a) mean serum Cₘₐₓ of about 359 μg/mL;
(b) mean serum Tₘₐₓ of about 0.13 days; or
(c) mean serum AUC_{Day0-Day28} of about 3486 μg·day/mL.

In some embodiments, three IV administrations of the composition comprising about 30 mg/kg of the anti-tau antibody, in which each administration is separated by about 28 days, achieves after the first administration one or more of:
(a) serum Cₘₐₓ of about 604 to about 874 μg/mL;
(b) serum Tₘₐₓ of about 0.06 to about 0.17 days; or
(c) serum AUC_{τ} of about 5458 to about 8320 μg·day/mL.

In some embodiments, three IV administrations of the composition comprising about 30 mg/kg of the anti-tau antibody, in which each administration is separated by about 28 days, achieves after the first administration one or more of:
(a) mean serum Cₘₐₓ of about 739 μg/mL;
(b) mean serum Tₘₐₓ of about 0.09 days; or
(c) mean serum AUC_{τ} of about 6889 μg·day/mL.

In some embodiments, three IV administrations of the composition comprising about 50 mg/kg of the anti-tau antibody, in which each administration is separated by about 28 days, achieves after the first administration one or more of:
(a) serum Cₘₐₓ of about 1039 to about 1305 μg/mL;
(b) serum Tₘₐₓ of about 0.06 to about 0.33 days; or
(c) serum AUC_{τ} of about 10,111 to about 11,751 μg·day/mL.

In some embodiments, three IV administrations of the composition comprising about 50 mg/kg of the anti-tau antibody, in which each administration is separated by about 28 days, achieves after the first administration one or more of:
(a) mean serum Cₘₐₓ of about 1172 μg/mL;
(b) mean serum Tₘₐₓ of about 0.17 days; or
(c) mean serum AUC_{τ} of about 10,931 μg·day/mL.

In some embodiments, three IV administrations of the composition comprising about 5 mg/kg of the anti-tau antibody, in which each administration is separated by about 28 days, achieves after the third administration one or more of:
(a) serum Cₘₐₓ of about 121 to about 173 μg/mL;
(b) serum Tₘₐₓ of about 0.05 to about 0.33 days; or
(c) serum AUC_{τ} of about 1112 to about 2080 μg.day/mL;
(d) serum AUCₗₐₛₜ of about 1641 to about 2965 μg·day/mL;
(e) serum AUC₂₈ of about 1716 to about 3060 μg·day/mL;
(f) serum t_{1/2} of about 13 to about 23.6 days; or
(g) serum CL of about 2.4 to about 4.2 mL/day/kg.

In some embodiments, three IV administrations of the composition comprising about 5 mg/kg of the anti-tau antibody, in which each administration is separated by about 28 days, achieves after the third administration one or more of:
(a) mean serum Cₘₐₓ of about 147 μg/mL;
(b) median serum Tₘₐₓ of about 0.11 days; or
(c) mean serum AUC_{τ} of about 1596 μg·day/mL;
(d) mean serum AUCₗₐₛₜ of about 2303 μg·day/mL;
(e) serum AUC_{∞} of about 2388 μg·day/mL;
(f) mean serum t_{1/2} of about 18.3 days; or
(g) serum CL of about 3.3 mL/day/kg.

In some embodiments, three IV administrations of the composition comprising about 5 mg/kg of the anti-tau antibody, in which each administration is separated by about 28 days, achieves one or more of:
(a) CSF/serum concentration ratio about 21 days after the first administration of about 0.20% to about 0.44%;
(b) CSF/serum concentration ratio about 42 days after the first administration of about 0.20% to 0.49%; or
(c) CSF/serum concentration ratio about 84 days after the first administration of about 0.22% to 0.72%.

In some embodiments, three IV administrations of the composition comprising about 5 mg/kg of the anti-tau antibody, in which each administration is separated by about 28 days, achieves one or more of:
(a) mean CSF/serum concentration ratio about 21 days after the first administration of about 0.30%;
(b) mean CSF/serum concentration ratio about 42 days after the first administration of about 0.31%; or
(c) mean CSF/serum concentration ratio about 84 days after the first administration of about 0.40%.

In some embodiments, three IV administrations of the composition comprising about 15 mg/kg of the anti-tau antibody, in which each administration is separated by about 28 days, achieves after the third administration one or more of:
(a) serum Cₘₐₓ of about 379 to about 519 μg/mL;
(b) serum Tₘₐₓ of about 0.05 to about 0.17 days; or
(c) serum AUC_{τ} of about 3875 to about 7211 μg·day/mL;
(d) serum AUCₗₐₛₜ of about 5557 to about 12,157 μg·day/mL;
(e) serum AUC₂₈ of about 5577 to about 14,129 μg·day/mL;
(f) serum t_{1/2} of about 19.4 to about 32.8 days; or
(g) serum CL of about 2.2 to about 3.6 mL/day/kg.

In some embodiments, three IV administrations of the composition comprising about 15 mg/kg of the anti-tau antibody, in which each administration is separated by about 28 days, achieves after the third administration one or more of:
(a) mean serum Cₘₐₓ of about 449 μg/mL;
(b) median serum Tₘₐₓ of about 0.17 days; or
(c) mean serum AUC_{τ} of about 5543 μg·day/mL;
(d) mean serum AUCₗₐₛₜ of about 8857 μg·day/mL;
(e) serum AUC₂₈ of about 9853 μg·day/mL;
(f) mean serum t_{1/2} of about 26.1 days; or
(g) serum CL of about 2.9 mL/day/kg.

In some embodiments, three IV administrations of the composition comprising about 15 mg/kg of the anti-tau antibody, in which each administration is separated by about 28 days, achieves one or more of:
(a) CSF/serum concentration ratio about 7 days after the first administration of about 0.11% to about 0.23%;
(b) CSF/serum concentration ratio about 14 days after the first administration of about 0.08% to about 0.58%;
(c) CSF/serum concentration ratio about 63 days after the first administration of about 0.75% to about 0.54%;
(d) CSF/serum concentration ratio about 70 days after the first administration of about 0.11% to about 0.44%; or
(e) CSF/serum concentration ratio about 84 days after the first administration of about 0.11% to 0.35%.

In some embodiments, three IV administrations of the composition comprising about 15 mg/kg of the anti-tau antibody, in which each administration is separated by about 28 days, achieves one or more of:
(a) mean CSF/serum concentration ratio about 7 days after the first administration of about 0.16%;
(b) mean CSF/serum concentration ratio about 14 days after the first administration of about 0.23%;
(c) mean CSF/serum concentration ratio about 63 days after the first administration of about 0.20%;
(d) mean CSF/serum concentration ratio about 70 days after the first administration of about 0.23%; or
(e) mean CSF/serum concentration ratio about 84 days after the first administration of about 0.20%.

In some embodiments, three IV administrations of the composition comprising about 30 mg/kg of the anti-tau antibody, in which each administration is separated by about 28 days, achieves after the third administration one or more of:
(a) serum Cₘₐₓ of about 657 to about 1187 μg/mL;
(b) serum Tₘₐₓ of about 0.07 to about 0.17 days; or
(c) serum AUC_{τ} of about 7190 to about 11,830 μg·day/mL;
(d) serum AUCₗₐₛₜ of about 9673 to about 20,587 μg·day/mL;
(e) serum AUC₂₈ of about 10,034 to about 22,244 μg·day/mL;
(f) serum t_{1/2} of about 19.9 to about 26.7 days; or
(g) serum CL of about 2.6 to about 3.9 mL/day/kg.

In some embodiments, three IV administrations of the composition comprising about 30 mg/kg of the anti-tau antibody, in which each administration is separated by about 28 days, achieves after the third administration one or more of:
(a) mean serum Cₘₐₓ of about 922 μg/mL;
(b) median serum Tₘₐₓ of about 0.17 days; or
(c) mean serum AUC_{τ} of about 9510 μg·day/mL;
(d) mean serum AUCₗₐₛₜ of about 15,130 μg·day/mL;
(e) serum AUC_{∞} of about 16,139 μg·day/mL;
(f) mean serum t_{1/2} of about 23.3 days; or
(g) serum CL of about 3.3 mL/day/kg.

In some embodiments, three IV administrations of the composition comprising about 30 mg/kg of the anti-tau antibody, in which each administration is separated by about 28 days, achieves one or more of:
(a) mean CSF/serum concentration ratio about 7 days after the first administration of about 0.18%;
(b) mean CSF/serum concentration ratio about 14 days after the first administration of about 0.26%;
(c) mean CSF/serum concentration ratio about 63 days after the first administration of about 0.25%;
(d) mean CSF/serum concentration ratio about 70 days after the first administration of about 0.29%; or
(e) mean CSF/serum concentration ratio about 84 days after the first administration of about 0.33%.

In some embodiments, three IV administrations of the composition comprising about 50 mg/kg of the anti-tau antibody, in which each administration is separated by about 28 days, achieves after the third administration one or more of:
(a) serum Cₘₐₓ of about 1146 to about 1550 μg/mL;
(b) serum Tₘₐₓ of about 0.07 to about 0.17 days; or
(c) serum AUC_{τ} of about 14,022 to about 17,222 μg·day/mL;
(d) serum AUCₗₐₛₜ of about 22,144 to about 27,486 μg·day/mL;
(e) serum AUC₂₈ of about 23,790 to about 30,366 μg·day/mL;
(f) serum t_{1/2} of about 24.3 to about 29.9 days; or
(g) serum CL of about 2.8 to about 3.6 mL/day/kg.

In some embodiments, three IV administrations of the composition comprising about 50 mg/kg of the anti-tau antibody, in which each administration is separated by about 28 days, achieves after the third administration one or more of:
(a) mean serum Cₘₐₓ of about 1348 μg/mL;
(b) median serum Tₘₐₓ of about 0.07 days; or
(c) mean serum AUC_{τ} of about 15,622 μg·day/mL;
(d) mean serum AUCₗₐₛₜ of about 24,815 μg·day/mL;
(e) serum AUC₂₈ of about 27,078 μg·day/mL;
(f) mean serum t_{1/2} of about 27.1 days; or
(g) serum CL of about 3.2 mL/day/kg.

In some embodiments, three IV administrations of the composition comprising about 50 mg/kg of the anti-tau antibody, in which each administration is separated by about 28 days, achieves one or more of:
(a) CSF/serum concentration ratio about 21 days after the first administration of about 0.23% to about 0.37%;
(b) CSF/serum concentration ratio about 42 days after the first administration of about 0.22% to about 0.45%; or
(c) CSF/serum concentration ratio about 84 days after the first administration of about 0.22% to 0.53%.

In some embodiments, three IV administrations of the composition comprising about 50 mg/kg of the anti-tau antibody, in which each administration is separated by about 28 days, achieves one or more of:
(a) mean CSF/serum concentration ratio about 21 days after the first administration of about 0.29%;
(b) mean CSF/serum concentration ratio about 42 days after the first administration of about 0.31%; or
(c) mean CSF/serum concentration ratio about 84 days after the first administration of about 0.34%.

### EXAMPLE

Embodiments of the present disclosure can be further defined by reference to the following non-limiting example. It will be apparent to those skilled in the art that many modifications, both to materials and methods, can be practiced without departing from the scope of the present disclosure.

### Pharmacokinetics and Safety of the Anti-Tau Antibody in Humans.

A two-part randomized, placebo-controlled, double-blind, single and multiple ascending dose study was performed to investigate safety and tolerability, pharmacokinetics, and pharmacodynamics of an anti-tau antibody of the present invention in healthy subjects and subjects with Alzheimer's Disease.

The anti-tau antibody used in the study was a humanized IgG1 monoclonal antibody comprising a heavy chain variable region having the amino acid sequence of SEQ ID NO: 25, and a light chain variable region having the amino acid sequence of SEQ ID NO: 26. The anti-tau antibody was supplied as a sterile, preservative-free liquid with a concentration of 50 mg/mL of the antibody in a solution composed of 10 mM histidine, 8.5% (w/v) sucrose, 0.04% (w/v) polysorbate 20, and 20 μg/mL EDTA, at a pH of 5.5.

### Methodology

The study consisted of two parts with nine total cohorts and up to eight subjects in each. Part 1 involved Cohorts 1-5, and Part 2 involved Cohorts A, B, D, and E.

Part 1 was a single ascending dose (SAD) study in healthy subjects to assess the safety, tolerability, pharmacokinetics, pharmacodynamics, and immunogenicity of the anti-tau antibody following single ascending IV doses of the anti-tau antibody. Single ascending IV doses ranging from 1 to 60 mg/kg of the anti-tau antibody or a placebo were administered to sequential cohorts of healthy subjects. Dosing for each cohort in Part 1 occurred over at least two days, with two subjects dosed on the first day (one receiving the placebo, one receiving the anti-tau antibody) and six subjects the following day(s) (one receiving the placebo, five receiving the anti-tau antibody). Consecutive subject dosing was separated by at least 60 minutes from the start of each infusion in each cohort.

Part 2 was a multiple ascending dose (MAD) study to assess the safety, tolerability, pharmacokinetics, and immunogenicity of the anti-tau antibody following multiple ascending IV doses of the anti-tau antibody, as well as assess pharmacodynamics in subjects with prodromal or mild Alzheimer's Disease and in healthy subjects. Two dose levels (5 mg/kg or 50 mg/kg) of the anti-tau antibody or placebo were evaluated in healthy subjects, and two dose levels (15 mg/kg or 30 mg/kg) of the anti-tau antibody or placebo were evaluated in subjects with prodromal or mild Alzheimer's Disease, as multiple ascending IV doses over a period of eight weeks (IV dosing occurred on Day 1, Day 29, and Day 57). If two or more subjects were available for dosing at the initiation of any given MAD cohort in Part 2, then sentinel dosing was done (as described for Part 1), with one subject receiving placebo and one subject receiving the prior to additional subjects being dosed.

Subjects were enrolled per the following inclusion and exclusion criteria:

General inclusion criteria:
Body mass index (BMI) between 18 and 35 kg/m², inclusive (BMI=weight/height²) and body weight greater than 40 kg but less than 110 kg at screening.
Women must not be of childbearing potential.

Specific inclusion criteria for Part 2:
Each potential subject enrolled in Part 2 must satisfy all of the following specific criteria in addition to the general criteria to be enrolled in the study.
Clinical Demential Rating global rating score of 0.5 or 1.0 at screening.
Must have a reliable informant (example, relative, partner, friend).
Must have CSF finding consistent with Alzheimer's Disease pathology.

General exclusion criteria
Any potential subject who meets any of the following criteria will be excluded from participating in the study.
History of or current liver or renal insufficiency; significant cardiac, vascular, pulmonary, gastrointestinal, endocrine, neurologic (including but not limited to neurodegenerative disease (excluding Alzheimer's Disease for Part 2), seizure disorders, transient ischemic attacks, etc.), hematologic (including coagulation disorders), rheumatologic, psychiatric, or metabolic disturbances, any inflammatory illness or any other illness that the Investigator considers should exclude the subject.
Relevant history of or current neurological disease (other than prodromal Alzheimer's Disease or mild Alzheimer's Disease for Part 2), which in the opinion of the Investigator may make interpretation of possible new neurological signs or symptoms difficult.
History of human immunodeficiency virus (HIV) antibody positive, or tests positive for HIV at screening (per screening evaluations).
History of hepatitis B surface antigen (HBsAg) or hepatitis C antibody (anti-Hepatitis C virus) positive, or other clinically active liver disease, or tests positive for HBsAg or anti-HCV at screening (per screening evaluations).

Specific exclusion criteria for Part 1
Mini-Mental State Examination (MMSE) score less than or equal to (<=) 27 at screening.

Specific exclusion criteria for Part 2
Evidence of brain disease, other than AD, that could explain the cognitive deficit (including, but not limited to, vascular encephalopathy or strokes, as imaged by cerebral magnetic resonance imaging (MRI)).

The subjects in Part 1 were male and female, 55 to 75 years of age, inclusive, and healthy. The subjects in Part 2 were male and female, 55 to 80 years of age inclusive, and included healthy subjects and subjects with prodromal or mild Alzheimer's Disease. Alzheimer's Disease subjects had a Clinical Demential Rating Global Score of 0.5 or 1.0 consistent with mild cognitive impairment (MCI; prodromal Alzheimer's Disease) or mild Alzheimer's Disease, respectively, as well as evidence of amyloid deposition and tauopathy as demonstrated by an abnormal CSF Aβ1-42 and elevated CSF p181tau.

Subjects were admitted to the unit on Day 1 and had a five-day/four-night inpatient period during their first IV administration for Part 1 and a 3 day/2-night inpatient period following their first IV administration for Part 2. For Part 2, on all subsequent dosing days (Day 29 and Day 57), subjects came to the unit during the morning on the day of IV administration and were discharged at least one hour post IV infusion at the discretion of the investigator if no safety issues were observed and all study visit assessments had been completed. On dosing days, subjects received the study intervention in the morning at least 30 minutes after the start of a standardized light breakfast (<500 calories).

For Part 1, dosages of 1 mg/kg, 3 mg/kg, 10 mg/kg, 30 mg/kg, and 60 mg/kg were administered for the various treatment arms. For Part 2, dosages of 5 mg/kg, 15 mg/kg, 30 mg/kg, and 50 mg/kg were administered for the various treatment arms. For both parts, the placebo was supplied as a 0.9% sodium chloride solution.

Following dosing and after completion of the inpatient phase, subjects from Part 1 returned to the study site for regular follow-up visits up to 13 weeks following dosing to assess safety, tolerability, pharmacokinetics (blood and CSF), immunogenicity (anti-drug antibodies [ADAs]), and pharmacodynamics (biomarker response; blood and CSF). Subjects from Part 2 returned for subsequent dose administrations on Day 29 and Day 57 and for regular follow-up visits up to 13 weeks following last dosing to assess safety, tolerability, pharmacokinetics (blood and CSF), immunogenicity (ADAs), and pharmacodynamics (biomarker response; blood and CSF).

Sampling schemes varied by cohort and were balanced across treatment groups to characterize the pharmacokinetic profile of the anti-tau antibody and assess the biomarker response.

Completion of the Day 92 (Week 13) visit for Part 1 and Day 148 (Week 21) visit for Part 2 constituted the end of participation in the study unless a CSF sample was collected at that visit. In that case, the subject had an additional safety follow-up visit at Day 106 (Week 15) for Part 1 or Day 162 (Week 23) for Part 2.

Safety and tolerability assessments included vital signs, safety labs, Mill of the brain, 12-lead electrocardiogram (ECG), and telemetry (Part 1 only).

Blood samples and CSF samples by single LPs (12 mL/puncture) were collected at regular time points (screening/baseline and post-dose) to assess peripheral and central pharmacokinetics, immunogenicity (i.e., ADAs), and pharmacodynamics (total, free and bound p217+tau, total tau, and/or p181tau) of the anti-tau antibody.

In Part 1, five CSF samples by single lumbar puncture were collected for each subject: a baseline sample collected at least two weeks prior to Day 1 following confirmation of eligibility, and at four post-dose time points separated by at least 12 days. In Part 2, four CSF samples by single lumbar puncture were collected for each subject: for healthy subjects, following confirmation of eligibility, a baseline sample was collected at least two weeks prior to Day 1, and for subjects with prodromal or mild Alzheimer's Disease, an initial CSF sample was used for confirming eligibility during the screening period. CSF was also collected in Part 2 at four post-dose time points separated by at least 12 days.

A mandatory separate pharmacogenomic (deoxyribonucleic acid [DNA]) blood sample was collected from all subjects. The goal of the pharmacogenomic component was to collect DNA to allow for the possible evaluation of genetic factors that may influence the pharmacokinetics, pharmacodynamics, safety, or tolerability of anti-tau antibody and pathways related to Alzheimer's Disease/tauopathy.

### Results

As shown in Tables 2 and 3, a total of 40 subjects (30 randomized to receiving the anti-tau antibody, 10 randomized to placebo) were enrolled in Part 1 of the study. Of the 40 subjects, one subject withdrew from the study (personal reasons) and 39 subjects completed the study. A total of 29 subjects (23 randomized to receiving the anti-tau antibody, six randomized to placebo) were enrolled in Part 2 of the study: 16 healthy subjects and 13 with prodromal or mild Alzheimer's Disease. Of the 29 subjects, one subject withdrew from the study (declined to undergo additional procedures) and 28 subjects completed the study.

TABLE 2
Number of subjects in Part 1 of the study.

| | | Anti-Tau Antibody | | | | |
|---|---|---|---|---|---|---|
| | | 1 | 3 | 10 | 30 | 60 |
| | Placebo | mg/kg | mg/kg | mg/kg | mg/kg | mg/kg |
| PK Analysis | 10 | 5 | 6 | 6 | 6 | 6 |
| Safety Analysis | 10 | 6 | 6 | 6 | 6 | 6 |

TABLE 3
Number of subjects in Part 2 of the study.

| | | | Anti-Tau Antibody | | | |
|---|---|---|---|---|---|---|
| | Placebo | | 15 mg/kg | 30 mg/kg | 5 mg/kg | 50 mg/kg |
| | AD | HP | (AD) | (AD) | (HS) | (HS) |
| PK Analysis | 2 | 4 | 6 | 4 | 6 | 6 |
| Safety Analysis | 2 | 4 | 6 | 5 | 6 | 6 |

AD = Alzheimer's Disease
HS = healthy subjects

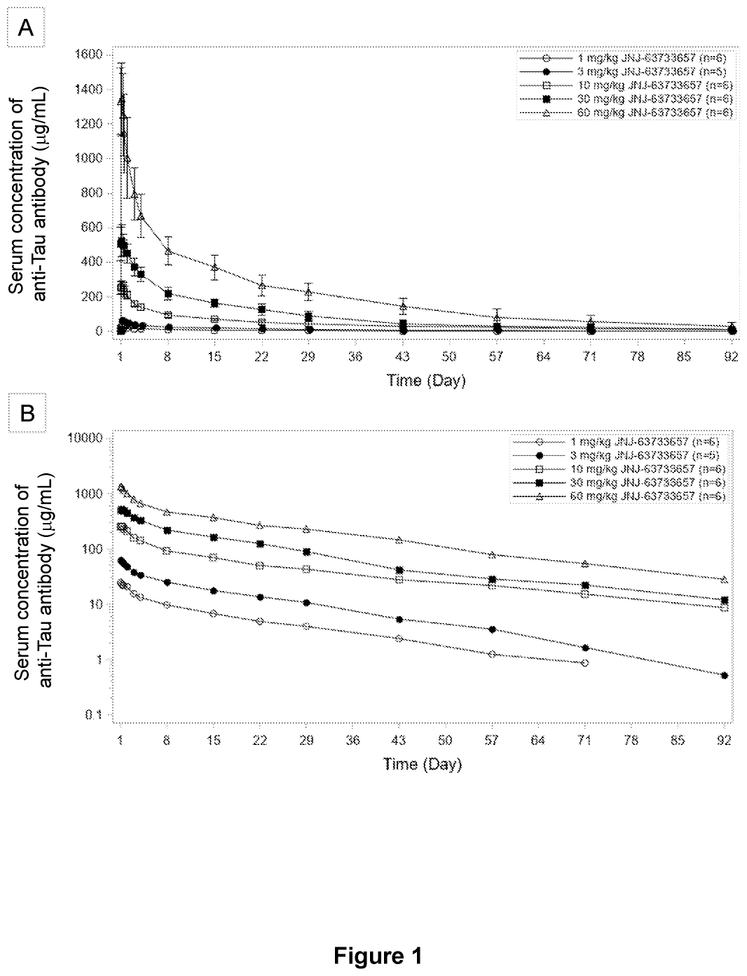

The pharmacokinetics results for Part 1 of the study are presented in Tables 4-6 and FIGS. 1-3. These results show that after administration of a single IV dose of the anti-tau antibody, median serum Tₘₐₓ ranged between 0.05 and 0.25 days after the start of administration across the different dosages. The mean serum t_{1/2} ranged between 18.1 days and 26.4 days following IV administration of the anti-tau antibody for the five cohorts. The mean serum CL ranged between 2.58 and 3.75 mL/day/kg and were comparable across the different dosages.

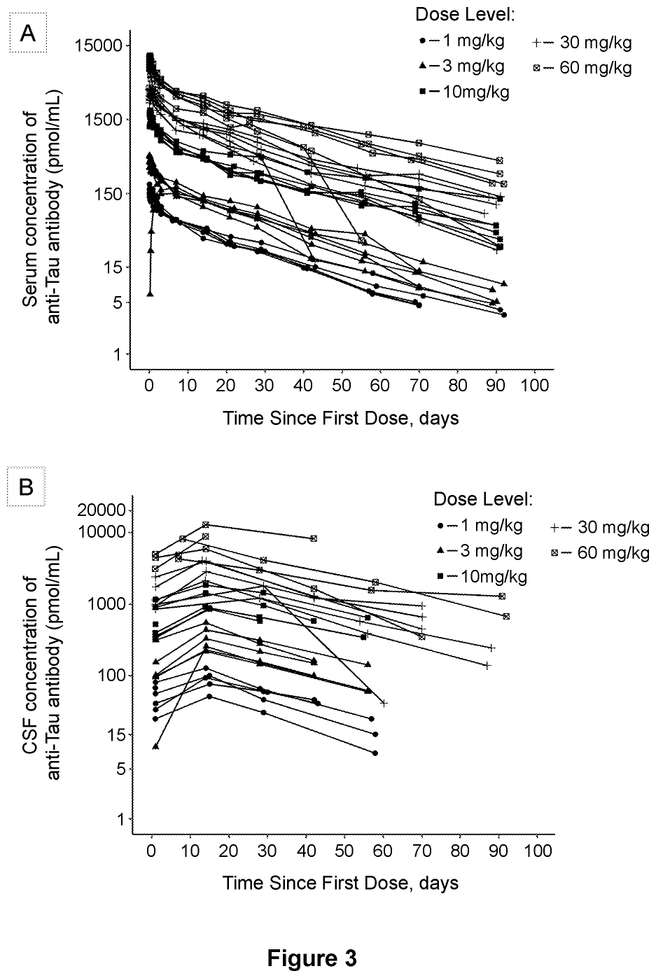

Mean serum Cmax, AUCiast, and AUC₂₈ increased with increasing dosages (see FIGS. 1 and 3). The mean values for the dose normalized (dn) serum pharmacokinetics parameters (C_{max,dn}, AUC_{last,dn}, and AUC_{∞,dn}) were comparable with increasing dosages, which suggests that the anti-tau antibody shows dose proportionality in serum within the dose range studied.

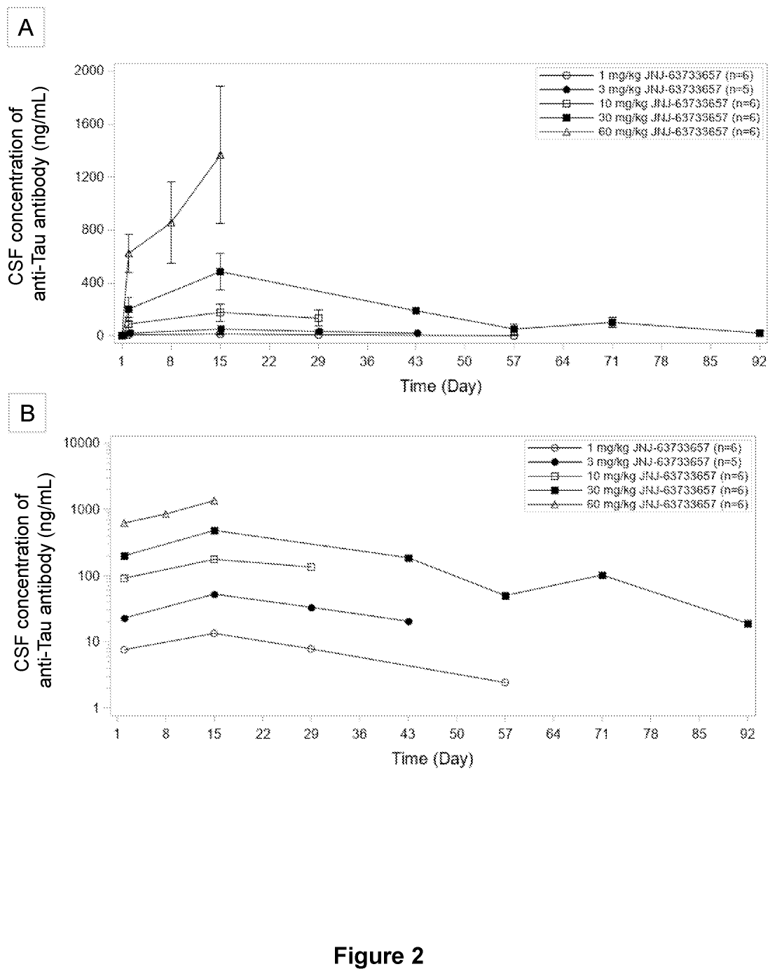

Mean CSF Cₘₐₓ, AUC_{Day29}, AUC_{Day43}, and AUC_{Day57} increased with increasing dosages (see FIGS. 2 and 3). In general, the mean values for the dose normalized CSF Cmax and AUCs were comparable, and the distributions of the individual values overlapped, which suggests a dose-proportional increase of the CSF Cₘₐₓ and AUCs over the studied dose range.

Following IV administration of the anti-tau antibody, the mean CSF/serum ratio on Day 2 ranged between 0.0345% and 0.0535% for all cohorts. From Day 8 onwards, the ratio of CSF/serum was comparable for all following sampling days and ranged between 0.191% and 0.450% for all cohorts.

TABLE 4
Serum pharmacokinetic results of the anti-tau antibody after administration of a single
IV dose of the anti-tau antibody in healthy subjects from Part 1 of the study.

| | Dosages of Anti-Tau Antibody | | | | |
|---|---|---|---|---|---|
| | 1 mg/kg | 3 mg/kg | 10 mg/kg | 30 mg/kg | 60 mg/kg |
| n | 6^{a} | 5 | 6 | 6^{b} | 6^{b} |
| Cₘₐₓ, μg/mL | 24.6 | 62.7 | 266 | 574 | 1387 |
| | (3.24) | (9.09) | (35.9) | (51.0) | (196) |
| Tₘₐₓ, day | 0.11 | 0.05 | 0.25 | 0.17 | 0.17 |
| | (0.050-0.50) | (0.04-0.17) | (0.05-0.33) | (0.06-0.50) | (0.06-0.92) |
| AUCₗₐₛₜ, | 322 | 819 | 3685 | 7499 | 17805 |
| μg · day/mL | (38.9) | (134) | (750) | (1578) | (4355) |
| AUC_{∞}, | 339 | 849 | 4062 | 8314 | 20447 |
| μg · day/mL | (38.5) | (144) | (1115) | (1762) | (3696) |
| λ_{z}, 1/day | 0.0378 | 0.0391 | 0.0278 | 0.0312 | 0.0348 |
| | (0.00679) | (0.00603) | (0.00662) | (0.00754) | (0.0127) |
| t_{1/2}, day | 18.9 | 18.1 | 26.4 | 23.1 | 21.8 |
| | (3.9) | (3.0) | (7.8) | (4.6) | (6.8) |
| CL, mL/day/kg | 2.97 | 3.60 | 2.58 | 3.75 | 3.03 |
| | (0.294) | (0.599) | (0.503) | (0.834) | (0.645) |
| C_{max, dn} | 24.7 | 21.0 | 26.6 | 19.1 | 23.1 |
| μg/mL/(mg/kg) | (3.17) | (3.15) | (3.59) | (1.70) | (3.27) |
| AUC_{last, dn} | 322 | 274 | 369 | 250 | 297 |
| μg · day/mL/(mg/kg) | (38.2) | (46.2) | (75.0) | (52.6) | (72.6) |
| AUC_{∞, dn} | 340 | 284 | 406 | 277 | 341 |
| μg · day/mL/(mg/kg) | (37.9) | (49.7) | (111) | (58.7) | (61.6) |

Note:
Pharmacokinetic parameters are presented as mean (SD), except Tₘₐₓ is presented as median(range).
^{a}n = 5 for AUCₗₐₛₜ, AUC_{∞}, λ_{z}, t_{1/2}, CL, AUC_{last, dn} and AUC_{∞, dn}
^{b}n = 5 for AUC_{∞}, λ_{z}, t_{1/2}, CL, and AUC_{∞, dn}

TABLE 5
CSF pharmacokinetic results of the anti-tau antibody after administration of a single IV
dose of the anti-tau antibody in healthy subjects from Part 1 of the study.

| | Dosages of Anti-Tau Antibody | | | | |
|---|---|---|---|---|---|
| | 1 mg/kg | 3 mg/kg | 10 mg/kg | 30 mg/kg | 60 mg/kg |
| n | 5^{y} | 5^{x} | 5^{y} | 6 | 6 |
| Cₘₐₓ, ng/mL | 13.4 | 52.7 | 176 | 397 | 1111 |
| | (4.21) | (21.1) | (65.3) | (173) | (474) |
| Tₘₐₓ, day | 14.93 | 14.06 | 14.00 | 14.13 | 10.97 |
| | (13.99-15.03) | (13.94-14.09) | (13.90-15.01) | (13.09-28.99) | (6.91-14.07) |
| AUC_{Day29}, | 296 | 1104 | 4276^{c} | — | — |
| ng · day/mL | (87.9) | (442) | (1911) | | |
| AUC_{Day43}, | — | 1524^{b} | — | 15420^{d} | — |
| ng · day/mL | | (610) | | (2819) | |
| AUC_{Day57}, | 437^{a} | — | — | 9658^{d} | — |
| ng · day/mL | (178) | | | (1778) | |
| AUC_{last, s1}, | — | 1524^{b} | — | 19468^{d} | — |
| ng · day/mL^{e} | | (610) | | (2441) | |
| AUC_{last, s2}, | 437^{a} | — | — | 10759^{d} | — |
| ng · day/mL^{f} | (178) | | | (1387) | |
| C_{max, dn}, | 13.5 | 17.6 | 17.6 | 13.2 | 18.5 |
| ng/mL/(mg/kg) | (4.37) | (7.17) | (6.53) | (5.77) | (7.89) |
| AUC_{Day29, dn}, | 297 | 370 | 428^{c} | — | — |
| ng · day/mL/(mg/kg) | (91.5) | (150) | (191) | | |
| AUC_{Day43, dn}, | — | 510^{b} | — | 514^{d} | — |
| ng · day/mL/(mg/kg) | | (207) | | (94.0) | |
| AUC_{Day57, dn}, | 440^{a} | — | — | 322^{d} | — |
| ng · day/mL/(mg/kg) | (186) | | | (59.3) | |
| AUC_{last, s1, dn}, | — | 510^{b} | — | 649^{d} | — |
| ng · day/mL/(mg/kg)^{e} | | (207) | | (81.4) | |
| AUC_{last,s2, dn}, | 440^{a} | — | — | 359^{d} | — |
| ng · day/mL/(mg/kg)^{f} | (186) | | | (46.2) | |

Note:
Each subject in a given cohort was randomized to 1 of 2 different LP sampling schemes.
Pharmacokinetic parameters are presented as mean (SD), except Tₘₐₓ is presented as median(range).
^{a}n = 3 for AUC_{Day57}, AUC_{last, s2}, AUC_{Day57, dn} and AUC_{last, s2, dn}
^{b}n = 3 for AUC_{Day43}, AUC_{last, s1}, AUC_{Day43, dn}, AUC_{last, s1, dn}
^{c}n = 4 for AUC_{Day29} and AUC_{Day29, dn}
^{d}n = 3 for AUC_{Day43}, AUC_{last, s1}, AUC_{Day43, dn}, AUC_{last, s1, dn}, AUC_{Day57}, AUC_{last, s2}, AUC_{Day57, dn} and AUC_{last, s2, dn}
^{e}AUCₗₐₛₜ of sampling scheme 1
^{f}AUCₗₐₛₜ of sampling scheme 2
^{x}One subject was excluded from standard output due to deviating PK profile.
^{y}One subject had a missing CSF sample around expected Cₘₐₓ. Cₘₐₓ and related parameters excluded from descriptive statistics

TABLE 6
Concentration ratios(CSF:serum) of the anti-tau antibody after administration of a
single IV dose of the anti-tau antibody in healthy subjects from Part 1 of the study.

| | Dosages of Anti-Tau Antibody | | | | |
|---|---|---|---|---|---|
| | 1 mg/kg | 3 mg/kg | 10 mg/kg | 30 mg/kg | 60 mg/kg |
| n | 6^{a} | 5^{b} | 6^{c} | 6^{d} | 3 |
| C_{Day2, CSF/serum} | 0.0345 | 0.0426 | 0.0390 | 0.0416 | 0.0535 |
| (%) | (0.0205-0.0581) | (0.0243-0.0748) | (0.0244-0.0624) | (0.0253-0.0683) | (0.0298-0.0961) |
| C_{Day8, CSF/serum} | — | — | — | — | 0.199 |
| (%) | | | | | (0.0920-0.431) |
| C_{Day15, CSF/serum} | 0.191 | 0.281 | 0.240 | 0.296 | 0.314 |
| (%) | (0.115-0.319) | (0.186-0.425) | (0.169-0.339) | (0.172-0.510) | (0.175-0.565) |
| C_{Day29, CSF/serum} | 0.191 | 0.301 | 0.278 | — | — |
| (%) | (0.126-0.289) | (0.226-0.400) | (0.195-0.397) | | |
| C_{Day43, CSF/serum} | — | 0.368 | — | 0.418 | — |
| (%) | | (0.169-0.802) | (0.156-1.12) | | |
| C_{Day57, CSF/serum} | 0.217 | — | — | — | — |
| (%) | (0.0640-0.738) | | | | |
| C_{Day71, CSF/serum} | — | — | — | 0.450 | — |
| (%) | | | | (0.158-1.28) | |

Note:
CSF:serum ratios are presented as geometric mean (95% confidential interval).
^{a}n = 5 for C_{Day15, CSF/serum}, C_{Day29, CSF/serum} and n = 3 for C_{Day57, CSF/serum}
^{b}n = 3 for C_{Day43, CSF/serum}
^{c}n = 5 for C_{Day15, CSF/serum} and n = 4 for C_{Day29, CSF/serum}
^{d}n = 4 for C_{Day15, CSF/serum} and n = 3 for C_{Day43, CSF/serum} and C_{Day71, CSF/serum}

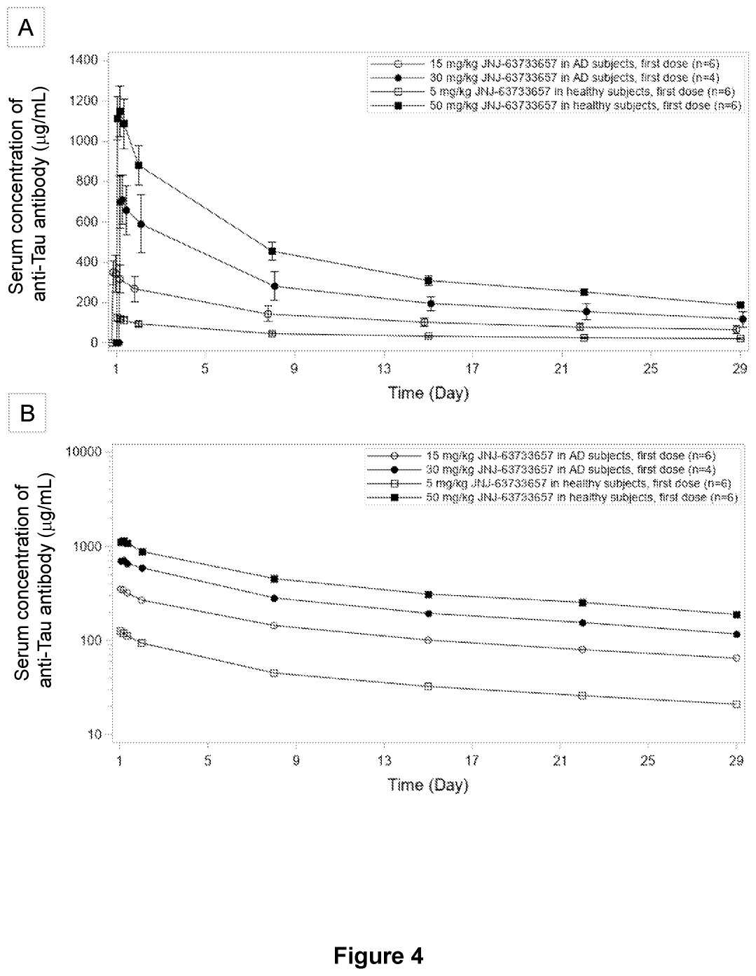

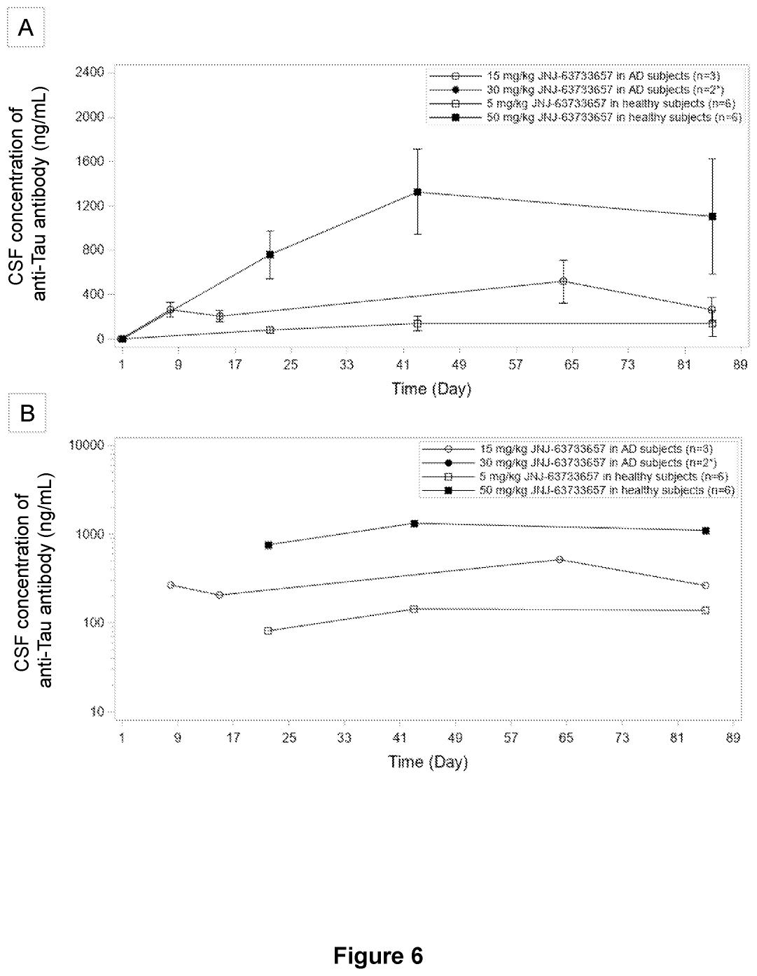

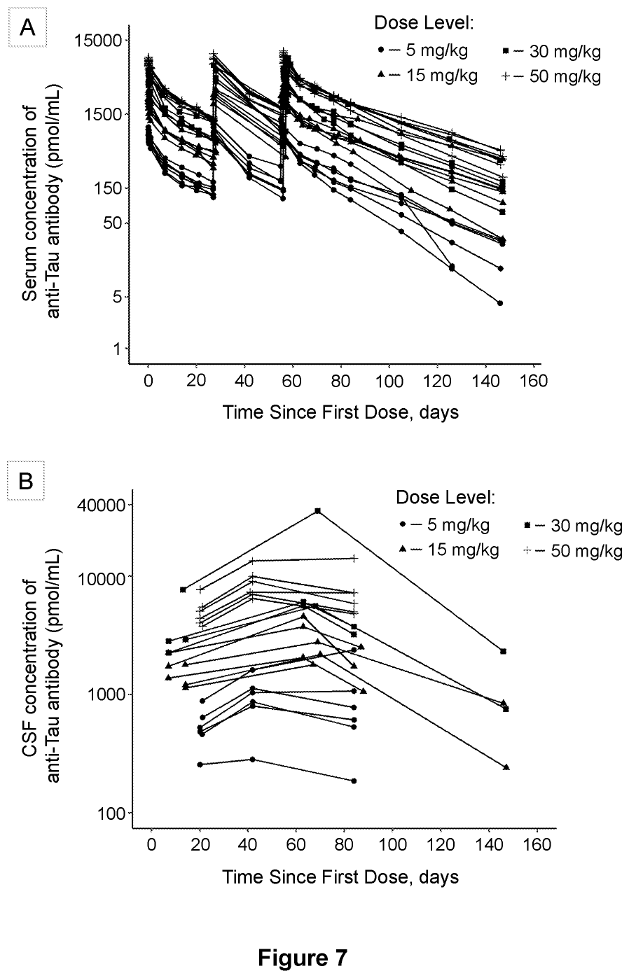

The pharmacokinetic results for Part 2 of the study are presented in Tables 7 and 8 and in FIGS. 4-7. These results show that, after administration of the first IV dose of the anti-tau antibody, median serum Tₘₐₓ ranged between 0.05 and 0.17 days after the start of administration. Mean serum Cₘₐₓ and AUC_{τ} increased with increasing dosages (see FIGS. 4 and 7) . The mean values for the dose normalized serum pharmacokinetic parameters (C_{max,dn} and AUC_{τ,dn}) were comparable with increasing dosages and between healthy subjects and subjects with Alzheimer's Disease, suggesting that the anti-tau antibody shows dose proportionality in serum within the dose range for both healthy and Alzheimer's Disease subjects and were similar to the results observed in Part 1.

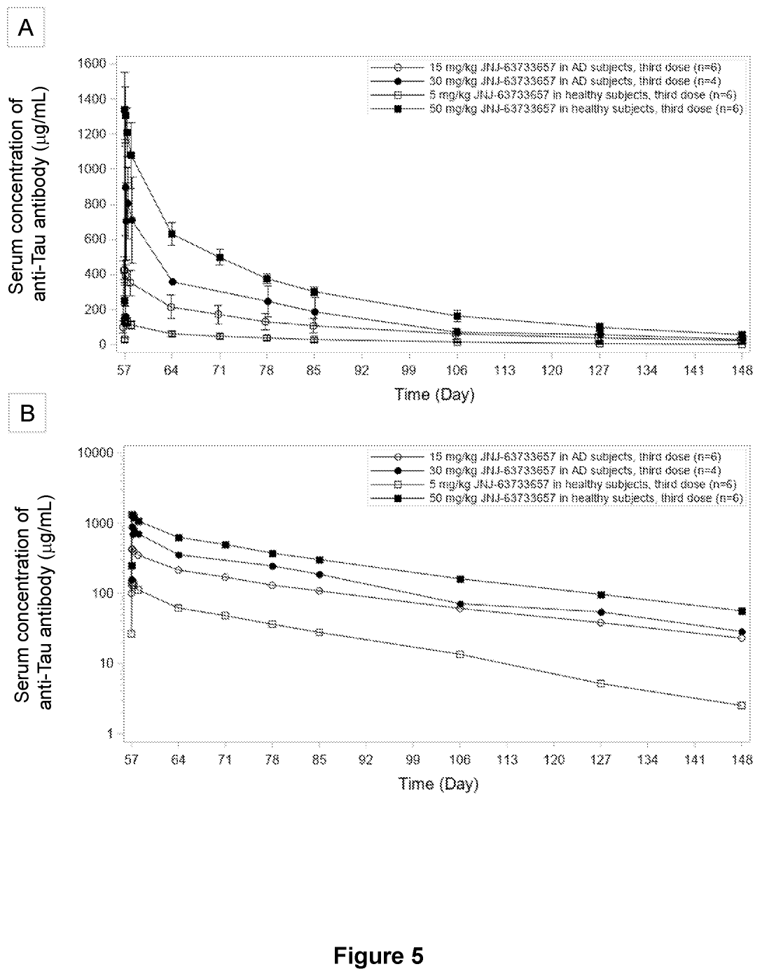

After administration of multiple IV doses of the anti-tau antibody, median serum Tₘₐₓ ranged between 0.07 and 0.17 days after the start of the third IV administration (see FIGS. 5 and 7), which generally corresponded to the sampling at the end of infusion and which is consistent with first dose.

The mean serum t_{1/2} ranged between 18.3 days and 27.1 days following the third IV administration of the anti-tau antibody. The mean serum CL ranged between 2.87 and 3.34 mL/day/kg and was comparable for the four cohorts and similar to the results in Part 1. The mean serum Vₛₛ ranged between 81.4 and 101 mL/kg and was comparable for the four cohorts.

Mean serum ratio of Cₘₐₓ of the first and third dose ranged between 1.15 and 1.26 and was comparable for all cohorts. Mean serum ratio of AUC_{τ} of the first and third dose, ranged between 1.39 and 1.59 μg·day/mL and was also comparable for all cohorts.

Following multiple IV administrations of the anti-tau antibody, the geometric mean and 95% CI of the CSF/serum ratio ranged between 0.164% (0.119-0.225%) and 0.401% (0.224-0.717%) for all cohorts across all sampling points and was comparable for all cohorts and sampling points. At Day 85, the geometric mean CSF/serum ratio was slightly higher for the cohorts of healthy subjects when compared to the cohorts of Alzheimer's Disease subjects (15 mg/kg and 30 mg/kg cohorts).

TABLE 7
Serum pharmacokinetic results of the anti-tau antibody after administration of the first
and third IV dose of the anti-tau antibody in healthy subjects from Part 2 of the study.

| Dosages of Anti-Tau Antibody | | | |
|---|---|---|---|
| 15 mg/kg (AD) | 30 mg/kg (AD) | 5 mg/kg (HS) | 50 mg/kg (HS) |

Day 1 (First Dose)

| | | | | |
|---|---|---|---|---|
| N | 6 | 5^{a} | 6 | 6 |
| Cₘₐₓ, μg/mL | 359 | 739 | 125 | 1172 |
| | (66.4) | (135) | (17.1) | (133) |
| Tₘₐₓ, day | 0.13 | 0.09 | 0.05 | 0.17 |
| | (0.05-0.17) | (0.06-0.17) | (0.04-0.33) | (0.06-0.33) |
| AUC_{τ}, μg · day/mL | 3486 | 6889 | 1130 | 10931 |
| | (830) | (1431) | (178) | (820) |
| C_{max, dn}, | 23.9 | 24.8 | 25.1 | 23.4 |
| μg/mL/(mg/kg) | (4.43) | (4.66) | (3.42) | (2.66) |
| AUC_{τ,dn}, | 232 | 231 | 226 | 219 |
| μg · day/mL/(mg/kg) | (55.3) | (50.1) | (35.7) | (16.4) |

Day 57(Third Dose)

| | | | | |
|---|---|---|---|---|
| N | 6 | 4 | 6 | 6 |
| Cₘₐₓ, μg/mL | 449 | 922 | 147 | 1348 |
| | (69.9) | (265) | (26.0) | (202) |
| Tₘₐₓ, day | 0.17 | 0.17 | 0.11 | 0.07 |
| | (0.05-0.17) | (0.07-0.17) | (0.05-0.33) | (0.07-0.17) |
| AUC_{τ}, μg · day/mL | 5543 | 9510 | 1596 | 15622 |
| | (1668) | (2320) | (484) | (1600) |
| AUCₗₐₛₜ, | 8857 | 15130 | 2303 | 24815 |
| μg · day/mL | (3300) | (5457) | (662) | (2671) |
| AUC_{∞}, | 9853 | 16139 | 2388 | 27078 |
| μg · day/mL | (4276) | (6105) | (672) | (3288) |
| λ_{z}, 1/day | 0.0283 | 0.0304 | 0.0408 | 0.0258 |
| | (0.00830) | (0.00515) | (0.0125) | (0.00294) |
| t_{1/2}, day | 26.1 | 23.3 | 18.3 | 27.1 |
| | (6.7) | (3.4) | (5.3) | (2.8) |
| CL, mL/day/kg | 2.87 | 3.27 | 3.34 | 3.23 |
| | (0.670) | (0.660) | (0.846) | (0.345) |
| Vₛₛ, mL/kg | 91.0 | 89.2 | 81.4 | 101 |
| | (13.3) | (20.5) | (23.7) | (20.0) |
| R_{Cmax, Day57/Day1} | 1.26 | 1.26 | 1.17 | 1.15 |
| | (0.134) | (0.137) | (0.113) | (0.120) |
| R_{AUC τ}, _{Day57/Day1} | 1.59 | 1.39 | 1.40 | 1.43 |
| | (0.268) | (0.190) | (0.270) | (0.0963) |
| C_{max, dn}, | 29.9 | 30.7 | 29.4 | 27.0 |
| μg/mL/(mg/kg) | (4.66) | (8.83) | (5.20) | (4.05) |
| AUC_{τ,dn}, | 370 | 317 | 319 | 312 |
| μg · day/mL/(mg/kg) | (111) | (77.3) | (96.7) | (32.0) |
| AUC_{last, dn,} | 590 | 504 | 461 | 496 |
| μg · day/mL/(mg/kg) | (220) | (182) | (132) | (53.4) |
| AUC_{∞, dn}, | 657 | 538 | 478 | 542 |
| μg · day/mL/(mg/kg) | (285) | (204) | (134) | (65.8) |

Note:
Pharmacokinetic parameters are presented as mean (SD), except Tₘₐₓ is presented as median(range).
AD = Alzheimer's Disease;
HS = healthy subjects.
^{a}n = 4 for AUC_{τ} and AUC_{τ,dn}

TABLE 8
Concentration ratios (CSF:serum) of the anti-tau antibody after administration of
multiple IV doses of the anti-tau antibody in healthy subjects from Part 2 of the study.

| | Dosages of Anti-Tau Antibody | | | |
|---|---|---|---|---|
| | 15 mg/kg (AD) | 30 mg/kg (AD) | 5 mg/kg (HS) | 50 mg/kg (HS) |
| N | 6 | 3 | 2^{a} | 6 |
| C_{Day8, CSF/serum} | — | 0.164 | 0.179; 0.124 | — |
| (%) | | (0.119-0.225) | | |
| C_{Day15, CSF/serum} | — | 0.225 | 0.263; 0.476 | — |
| (%) | | (0.0889-0.571) | | |
| C_{Day22, CSF/serum} | 0.299 | — | — | 0.291 |
| (%) | (0.205-0.434) | | | (0.232-0.366) |
| C_{Day43, CSF/serum} | 0.311 | — | — | 0.314 |
| (%) | (0.220-0.448) | | | (0.200-0.485) |
| C_{Day64, CSF/serum} | — | 0.201 | 0.253; 0.233 | — |
| (%) | | (0.0752-0.537) | | |
| C_{Day71, CSF/serum} | — | 0.228 | 0.287; 1.13 | — |
| (%) | | (0.119-0.435) | | |
| C_{Day85, CSF/serum} | 0.401 | 0.200 | 0.333; 0.414 | 0.339 |
| (%) | (0.224-0.717) | (0.116-0.348) | | (0.220-0.524) |

Note:
CSF:serum ratios are presented as geometric mean (95% confidential interval).
AD = Alzheimer's Disease;
HS = healthy subjects.
Ratios of CSF to corresponding serum concentration of the anti-tau antibody at Day X (C_{Dayx, CSF/serum})
^{a}n = 2 therefore mean ratio is not reported but individual values are presented for AD-30 mg/kg

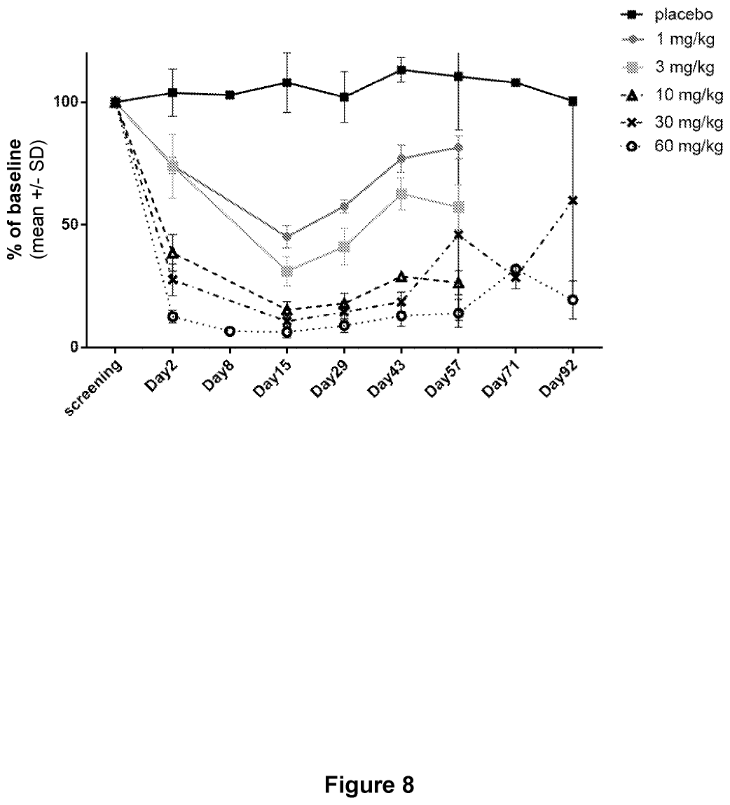

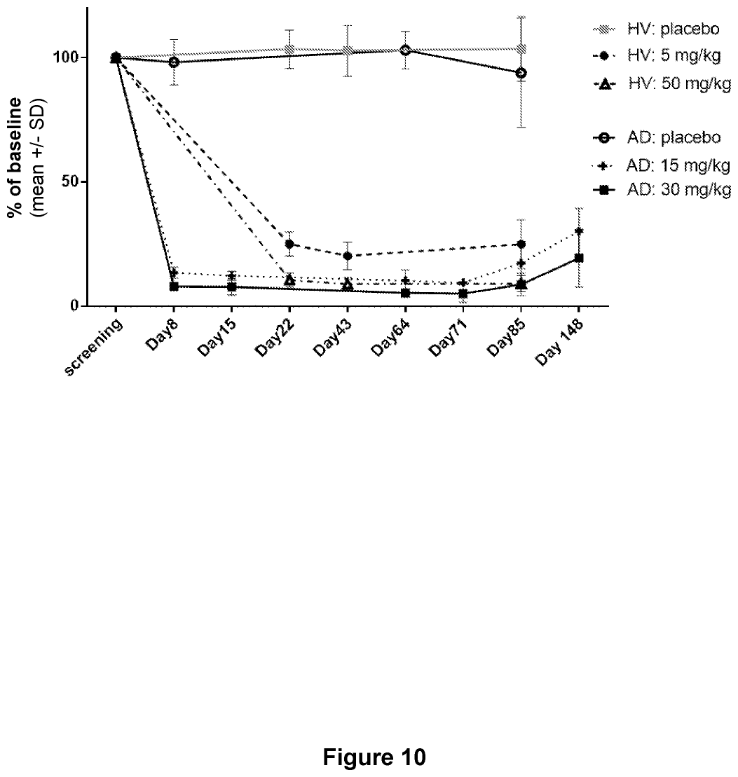

The pharmacodynamic results of the study are presented in FIGS. 8-15. The results indicate that there was a dose-dependent decrease in free and total CSF p217+tau observed in subjects in both Part 1 and Part 2. Maximum reduction was seen in Part 1 at eight days post-dose and began to rebound, but did not return to baseline levels, by 56 days post-dose. With monthly dosing in Part 2, no rebound in CSF p217+tau level was observed over at least 85 days (28 days after the last dose), suggesting sustained maximal impact of the anti-tau antibody.

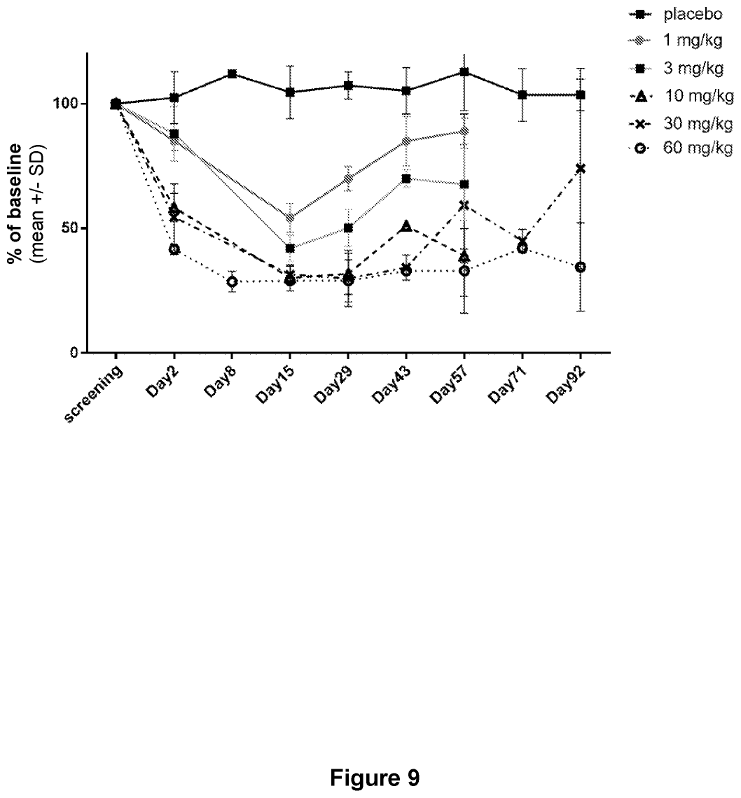

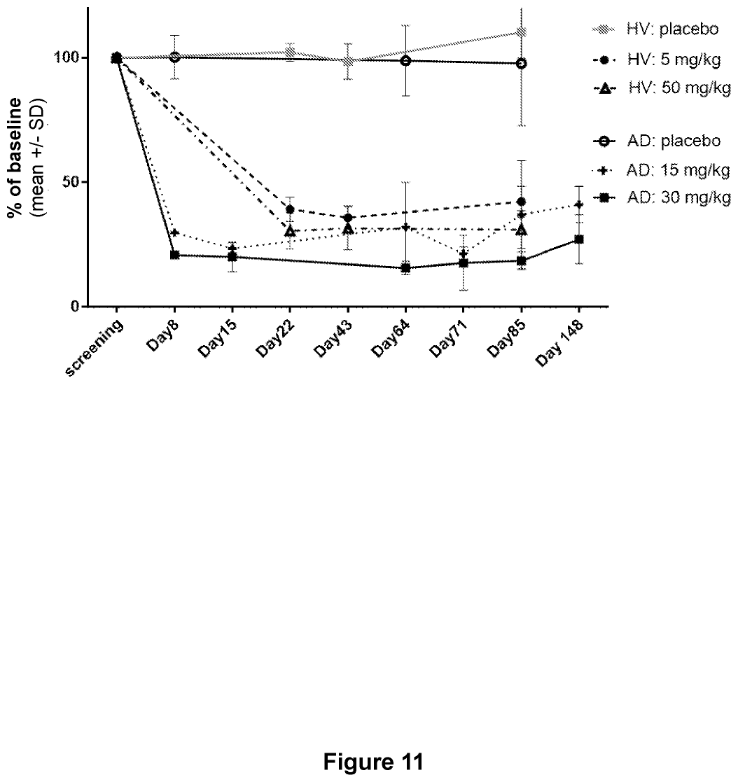

In Part 1, the percent of baseline total p217+tau at 14 days post-dose was 105% with placebo and 54%, 42%, 30%, 32%, and 29% with the 1 mg/kg, 3 mg/kg, 10 mg/kg, 30 mg/kg, and 60 mg/kg dosages for the anti-tau antibody, respectively (see FIG. 9). In Part 2, the percent of baseline total p217+tau at Day 71 or Day 85 was 110% with placebo, 42% with the 5 mg/kg dose and 31% with the 50 mg/kg dose in healthy subjects; and was 98% with placebo, 29% with the 15 mg/kg dose, and 18% with the 30 mg/kg dose in Alzheimer's Disease subjects (see FIG. 11).

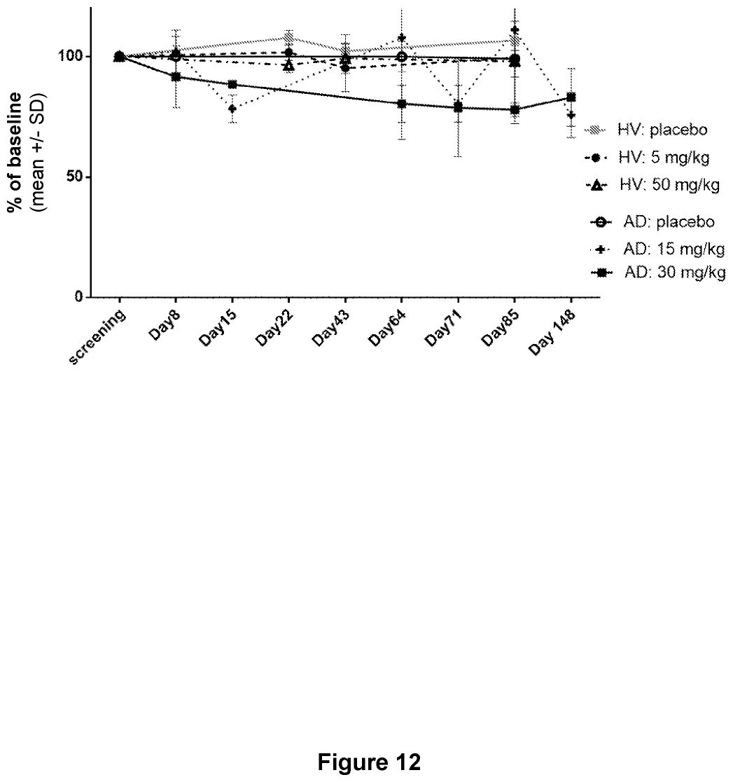

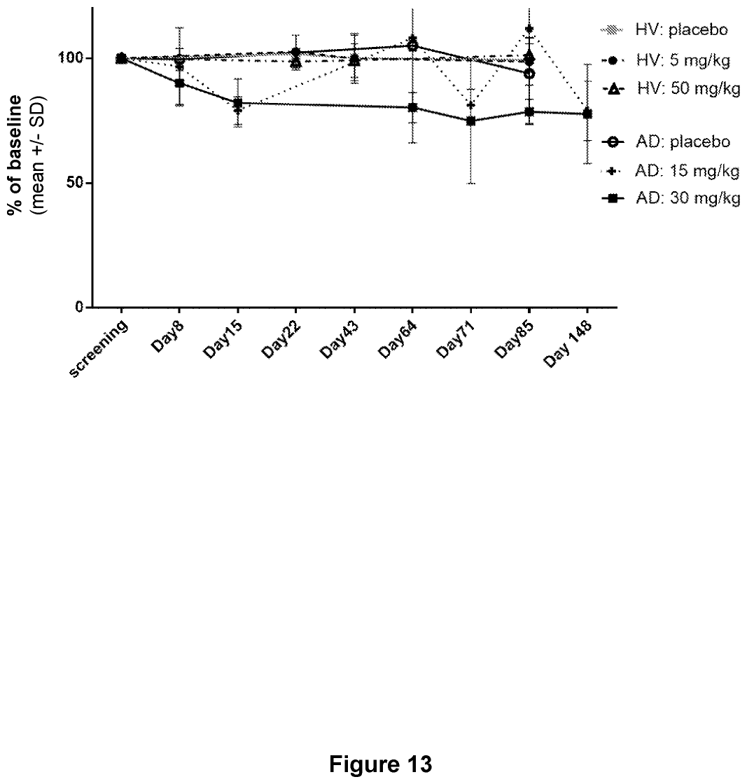

No change in total tau or p181tau was observed in healthy subjects from Part 1 or Part 2, while Alzheimer's Disease subjects exhibited a reduction in both total tau and p181tau (see FIGS. 12 and 13). The percent of baseline total tau at Day 71 or Day 85 was 107% with placebo, 99% with the 5 mg/kg dose and 98% with the 50 mg/kg dose in healthy subjects; and was 99% with placebo, 96% with the 15 mg/kg dose , and 78% with the 30 mg/kg dose in Alzheimer's Disease subjects (see FIG. 12). The percent of baseline p181tau at Day 71 or Day 85 was 99% with placebo, 99% with the 5 mg/kg dose, and 101% with the 50 mg/kg dose in healthy subjects; and was 94% with placebo, 96% with the 15 mg/kg dose, and 77% with the 30 mg/kg dose in Alzheimer's Disease subjects (see FIG. 13). The reductions of total tau and p181tau in Alzheimer's Disease subjects but not in healthy subjects may be due to a higher percentage of all tau species containing p217+in Alzheimer's Disease subjects (10-35%) as compared with healthy subjects (3-8%).

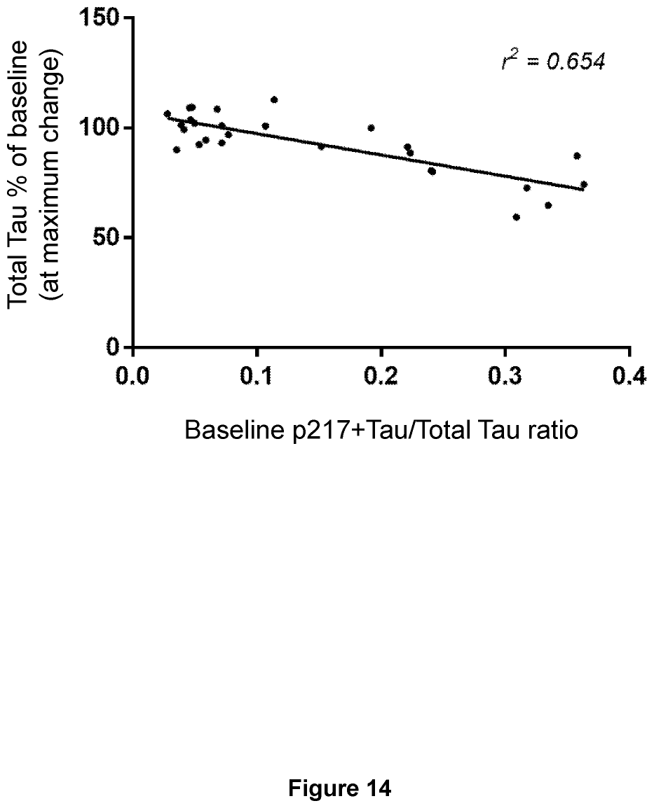

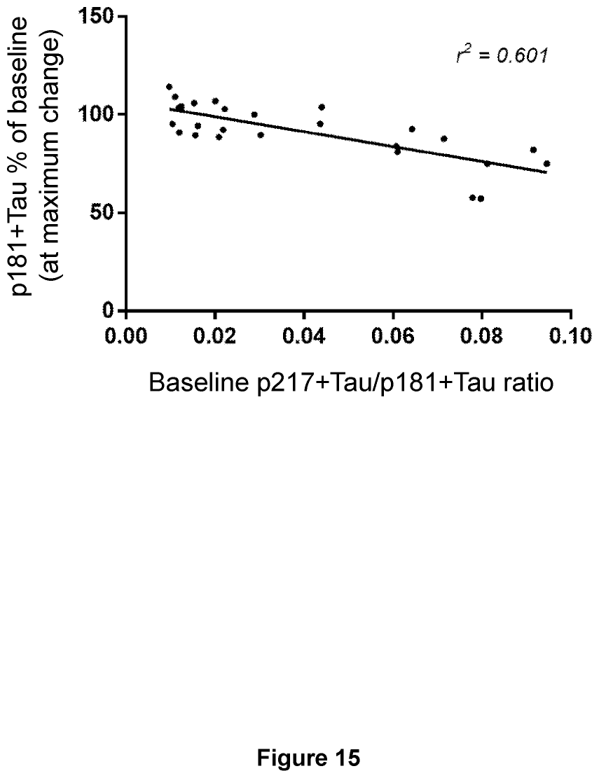

There was an observed correlation (r²=0.654 and 0.601) between the percent of tau that contains p21′7+and the magnitude of the total tau or p181tau reduction, which indicates that a robust impact on p217+tau is evident in the total tau and p181tau measurements in Alzheimer's Disease subjects (see FIGS. 14 and 15).

Among the 53 subjects receiving the anti-tau antibody (in Part 1 and Part 2), 11 subjects (eight subjects in Part 1 and three subjects in Part 2) were observed to be positive for treatment-emergent ADAs. Six subjects had a peak titer of 22.5, two subjects had a peak titer of 45, one subject had a peak titer of 180, and two subjects had a peak titer of 360. No treatment-emergent ADA was reported in the Alzheimer's Disease subject cohorts. The number of subjects with titers were higher at later timepoints and higher titers were detected at the later timepoints.

There were no deaths reported during the study and no early terminations due to treatment-emergent adverse events (TEAEs). Serious adverse events were reported in two subjects: in Part 1, a healthy subject treated with placebo experienced post lumbar puncture syndrome/suspected post spinal headache and hypertension; and in Part 2, an Alzheimer's Disease subject treated with the 15 mg/kg anti-tau antibody dose experienced renal neoplasm, although this adverse event was not considered related to the treatment with the anti-tau antibody.

All subjects who received at least one dose of study intervention were included in the safety analysis set. In Part 1 of the study, 24 (80%) of the 30 subjects treated with the anti-tau antibody reported one or more adverse events (AEs); 50% of subjects treated with 1 mg/kg, 66.7% of subjects treated with 3 mg/kg, 100% of subjects treated with 10 mg/kg, 83.3% of subjects treated with 30 mg/kg, and 100% of subjects treated with 60 mg/kg. Of the ten subjects treated with placebo, eight (80%) reported one or more AEs.

In Part 1 of the study, the most commonly reported TEAEs (>20% of subjects) were post lumbar puncture syndrome in subjects who received the 1 mg/kg dose of the anti-tau antibody; post lumbar puncture syndrome, hypercholesterolemia, headache, nausea, and hot flush in subjects who received the 10 mg/kg dose of the anti-tau antibody; hepatic enzyme increase in subjects who received the 30 mg/kg dose of the anti-tau antibody; headache, hypercholesterolemia, post lumbar puncture syndrome, procedural pain, muscle spasms, and neck pain in subjects who received the 60 mg/kg dose of the anti-tau antibody; and headache and back pain in subjects who received placebo. No TEAEs were reported in more than one subject who received the 3 mg/kg dose of the anti-tau antibody.

In Part 2 of the study, 20 (87%) of the 23 subjects treated with the anti-tau antibody reported one or more AEs; 66.7% of subjects treated with 5 mg/kg, 83.3% of subjects treated with 15 mg/kg, 100% of subjects treated with 30 mg/kg, and 100% of subjects treated with 50 mg/kg. Of the six subjects treated with placebo, five (83.3%) reported one or more AEs.

In Part 2 of the study, the most commonly reported TEAEs (>20% of subjects) were back pain and headache in subjects who received the 15 mg/kg dose of the anti-tau antibody; headache and post lumbar puncture syndrome in subjects who received the 50 mg/kg dose of the anti-tau antibody; and headache and fatigue in subjects who received placebo. No TEAEs were reported in more than one subject who received the 5 mg/kg dose or the 30 mg/kg dose of the anti-tau antibody.

No clinically important abnormalities were observed in any of the laboratory values, vital sign parameters, or brain MRIs.

### REFERENCES

Abhinandan K R and Martin ACR. Analysis and improvements to Kabat and structurally correct numbering of antibody variable domains. Mol. Immunol. 45: 3832-3839 (2008).

Almagro J C. Identification of differences in the specificity-determining residues of antibodies that recognize antigens of different size: implications for the rational design of antibody repertoires. J. Mol. Recognit. 17: 132-143 (2004).

Alonso A, et al. Hyperphosphorylation induces self-assembly of tau into tangles of paired helical filaments/straight filaments. Proc. Natl. Acad. Sci. USA. 98: 6923-6928 (2001).

Asuni A A, et al. Immunotherapy targeting pathological tau conformers in a tangle mouse model reduces brain pathology with associated functional improvements. J. Neurosci. 27: 9115-9129 (2007).

Bierer L M, et al. Neocortical neurofibrillary tangles correlate with dementia severity in Alzheimer's disease. Arch. Neurol. 52: 81-88 (1995).

Boutajangout A, et al. Passive immunization targeting pathological phospho-tau protein in a mouse model reduces functional decline and clears tau aggregates from the brain. J. Neurochem. 118: 658-667 (2011).

Braak H and Braak E. Neuropathological stageing of Alzheimer-related changes. Acta Neuropathol. 82: 239-259 (1991).

Berg L. Clinical Dementia Rating (CDR). Psychopharmacol. Bull. 24: 637-639 (1988).

Brunden KR, et al. Advances in tau-focused drug discovery for Alzheimer's disease and related tauopathies. Nat. Rev. Drug Discov. 8: 783-793 (2009).

Butner K A and Kirschner M W. Tau protein binds to microtubules through a flexible array of distributed weak sites. J. Cell. Biol. 115: 717-730 (1991).

Chothia C. and Lesk M. Canonical structures for the hypervariable regions of immunoglobulins. J. Mol. Biol. 196: 901-917 (1987).

Delacourte A. The molecular parameters of tau pathology. Tau as a killer and a witness. Adv. Exp. Med. Biol. 487: 5-19 (2001).

Dubois B, et al. Advancing research diagnostic criteria for Alzheimer's disease: the IWG-2 criteria. Lancet Neurol. 13: 614-629 (2014).

Dubois B, et al. Preclinical Alzheimer's disease: definition, natural history, and diagnostic criteria. Alzheimers Dement. 12: 292-323 (2016).

Fishwild D M, et al. High-avidity human IgG kappa monoclonal antibodies from a novel strain of minilocus transgenic mice. Nat. Biotechnol. 14: 845-51 (1996).

Friedhoff P, et al. Structure of tau protein and assembly into paired helical filaments. Biochim. Biophys. Acta. 1502: 122-132 (2000).

Goedert M, et al. Neurofibrillary tangles and beta-amyloid deposits in Alzheimer's disease. Curr. Opin. Neurobiol. 1: 441-447 (1991).

Hanger D P, et al. Tau phosphorylation: the therapeutic challenge for neurodegenerative disease. Trends Mol. Med. 15: 112-119 (2009).

Jack Jr. J R, et al. NIA-AA Research Framework: Toward a biological definition of Alzheimer's disease. Alzheimers Dement. 14: 535-562 (2018).

Knappik A., et al. Fully synthetic human combinatorial antibody libraries (HuCAL) based on modular consensus frameworks and CDRs randomized with trinucleotides. J. Mol. Biol. 296: 57-86 (2000).

Köhler G and Milstein C. Continuous cultures of fused cells secreting antibody of predefined specificity. Nature. 256: 495-497 (1975).

Krebs B, et al. High-throughput generation and engineering of recombinant human antibodies. J. Immunol. Methods. 254: 67-84 (2001).

Lefranc M P, et al. IMGT unique numbering for immunoglobulin and T cell receptor variable domains and Ig superfamily V-like domains. Dev. Comp. Immunol. 27: 55-77 (2003).

Lonberg N, et al. Antigen-specific human antibodies from mice comprising four distinct genetic modifications. Nature. 368: 856-859 (1994).

Martin A C R. Antibody Engineering. Kontermann R and Dubel S eds., Springer-Verlag, Berlin, 2: 33-51 (2010).

Mendez M J, et al. Functional transplant of megabase human immunoglobulin loci recapitulates human antibody response in mice. Nat. Genet. 15: 146-56 (1997).

Remington's Pharmaceutical Science. Osol A and Hoover J E eds., Mack Publishing Company, Easton, Pa. (15th ed. 1980).

Schroeder S K, et al. Tau-directed immunotherapy: a promising strategy for treating Alzheimer's disease and other tauopathies. J. Neuroimmune Pharmacol. 11: 9-25 (2016).

Shi L, et al. De novo selection of high-affinity antibodies from synthetic fab libraries displayed on phage as pIX fusion proteins. J. Mol. Biol. 397: 385-396 (2010).

Sigurdsson E M. Tau immunotherapy. Neurodegener. Dis. 16: 34-38 (2016).

Wu TT and Kabat E. An analysis of the sequences of the variable regions of Bence Jones proteins and myeloma light chains and their implications for antibody complementarity. J. Exp. Med. 132: 211-250 (1970).

1. A method of reducing total cerebrospinal fluid p217+tau in a subject in need thereof, the method comprising administering to the subject a composition comprising a pharmaceutically acceptable carrier and about 1 mg/kg to about 60 mg/kg per dose of a monoclonal antibody,
wherein the monoclonal antibody comprises a heavy chain variable complementarity-determining region (CDR) 1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15.

2. A method of reducing free cerebrospinal fluid p217+tau in a subject in need thereof, the method comprising administering to the subject a composition comprising a pharmaceutically acceptable carrier and about 1 mg/kg to about 60 mg/kg per dose of a monoclonal antibody,
wherein the monoclonal antibody comprises a heavy chain variable complementarity-determining region (CDR) 1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15.

3. A method of reducing total cerebrospinal fluid tau in a subject in need thereof, the method comprising administering to the subject a composition comprising a pharmaceutically acceptable carrier and about 1 mg/kg to about 60 mg/kg per dose of a monoclonal antibody,
wherein the monoclonal antibody comprises a heavy chain variable complementarity-determining region (CDR) 1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15.

4. A method of reducing cerebrospinal fluid p181tau in a subject in need thereof, the method comprising administering to the subject a composition comprising a pharmaceutically acceptable carrier and about 1 mg/kg to about 60 mg/kg per dose of a monoclonal antibody,
wherein the monoclonal antibody comprises a heavy chain variable complementarity-determining region (CDR) 1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15.

5. The method of any one of claims 1-4, wherein the monoclonal antibody comprises a heavy chain variable CDR1 having the amino acid sequence of SEQ ID NO: 1, a heavy chain variable CDR2 having the amino acid sequence of SEQ ID NO: 2, a heavy chain variable CDR3 having the amino acid sequence of SEQ ID NO: 3, a light chain variable CDR1 having the amino acid sequence of SEQ ID NO: 13, a light chain variable CDR2 having the amino acid sequence of SEQ ID NO: 14, and a light chain variable CDR3 having the amino acid sequence of SEQ ID NO: 15.

6. The method of any one of claims 1-5, wherein the monoclonal antibody comprises a heavy chain variable region comprising the amino acid sequence of SEQ ID NO: 25, and a light chain variable region comprising the amino acid sequence of SEQ ID NO:

26.

7. The method of any one of claims 1-6, wherein the monoclonal antibody comprises a heavy chain variable region having the amino acid sequence of SEQ ID NO: 25, and a light chain variable region having the amino acid sequence of SEQ ID NO: 26.

8. The method of any one of claims 1-7, wherein the monoclonal antibody comprises a heavy chain comprising the amino acid sequence of SEQ ID NO: 27, and a light chain comprising the amino acid sequence of SEQ ID NO: 28.

9. The method of any one of claims 1-8, wherein the monoclonal antibody comprises a heavy chain having the amino acid sequence of SEQ ID NO: 27, and a light chain having the amino acid sequence of SEQ ID NO: 28.

10. The method of any one of claims 1-9, wherein the composition further comprises histidine, sucrose, polysorbate 20, and ethylenediamine tetra-acetic acid.

11. The method of any one of claims 1-10, wherein the composition has a pH of about 5-6.

12. The method of any one of claims 1-11, comprising administering to the subject the composition comprising about 10 mg/kg to about 40 mg/kg per dose of the monoclonal antibody.

13. The method of any one of claims 1-11, comprising administering to the subject the composition comprising about 20 mg/kg to about 60 mg/kg per dose of the monoclonal antibody.

14. The method of any one of claims 1-11, comprising administering to the subject the composition comprising about 40 mg/kg to about 60 mg/kg per dose of the monoclonal antibody.

15. The method of any one of claims 1-11, comprising administering to the subject the composition comprising about 1 mg/kg, 3 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, or any value in between, per dose of the monoclonal antibody.

16. The method of any one of claims 1-15, wherein the composition is administered by intravenous infusion.

17. The method of any one of claims 1-16, wherein the composition is administered as more than one dose.

18. The method of claim 17, wherein the administration of each dose is separated by a period of about 4 weeks.

19. The method of any one of claims 1-18, wherein the administration of the monoclonal antibody results in a median serum Tₘₐₓ of the monoclonal antibody of about 0.05 days to about 0.25 days after administration.

20. The method of any one of claims 1-19, wherein the administration of the monoclonal antibody results in a median serum T_{1/2} of the monoclonal antibody about 18 days to about 27 days after administration.

21. The method of any one of claims 1-20, wherein the subject in need of a treatment of Alzheimer's Disease.

22. The method of claim 21, wherein the subject is in need of a treatment of early Alzheimer's Disease, mild cognitive impairment (MCI) due to Alzheimer's Disease, or mild to moderate Alzheimer's Disease.
